(19) United States
(12) Patent Application Publication
(10) Pub. No.: US 2025/0108125 A1
(43) Pub. Date: Apr. 3, 2025

(54) POLYMER NANOPARTICLE COMPOSITIONS FOR NON-VIRAL GENE DELIVERY TO THE CENTRAL NERVOUS SYSTEM

(71) Applicant: BATTELLE MEMORIAL INSTITUTE, Columbus, OH (US)

(72) Inventors: Kenneth R. SIMS, JR., Delaware, OH (US); Joseph GEERS, Columbus, OH (US); Danielle J. HUK, Hilliard, OH (US); Emma K. Schmitz, Columbus, OH (US)

(21) Appl. No.: 18/902,135

(22) Filed: Sep. 30, 2024

Related U.S. Application Data
(60) Provisional application No. 63/587,040, filed on Sep. 29, 2023.

(51) Int. Cl. A61K 47/69; A61K 31/7088; A61K 48/00; B82Y 5/00; C08F 293/00

(52) U.S. Cl. CPC A61K 47/6933; A61K 31/7088; A61K 48/0041; C08F 293/00; B82Y 5/00

(57) ABSTRACT

The disclosure relates to block copolymer nanoparticles for in vivo therapeutic delivery, and methods therefor. More particularly, the invention relates to polymer nanoparticles, such as reversible addition-fragmentation chain transfer (RAFT) polymer compositions, for delivering nucleotides to the central nervous system.

### RELATED APPLICATIONS

This application claims the benefit of U.S. Provisional Application No. 63/587,040, filed Sep. 29, 2023, the entire disclosure of which is incorporated herein by reference.

### BACKGROUND

Genetic medicines (including gene therapy, gene silencing, splicing regulators, and nuclease based gene editors) are poised to produce revolutionary treatments, including vaccines, infectious disease treatments, antimicrobial treatments, antiviral treatments, and most notably, genetic disease treatments. However, the in vivo delivery of these genetic medicine payloads to the specific tissues and cells that need to be treated, while avoiding tissues and cells that can reduce the efficacy or safety of the genetic medicine, poses a significant challenge. Additional challenges include the ability to deliver large genetic payloads or multiple payloads. Adeno-associated viruses (AAVs) are the most widely used tool for genetic medicine delivery, but AAVs are not able to deliver large genetic payloads or multiple payloads (such as the clustered regularly interspaced short palindromic repeats (CRISPR)/Cas9 system), and they sometimes trigger unwanted immune responses, including the generation of anti-AAV antibodies, a cell mediated response. Some of the immune responses caused by AAV in patients are potentially fatal immune responses.

Therapeutics based on the CRISPR/Cas9 system have an exceptional potential to treat a number of genetic diseases due to the capability of this system for precise and programmable gene editing. Gene editing and repair using the CRISPR/Cas9 system has two main mechanisms, including non-homologous end joining (NHEJ) which repairs the site of cut by inducing random indel mutation, and homology-directed repair (HDR), which repairs the cut site based on a pre-existing template. Because a pre-designed template can be used for HDR-directed repair, therapies based on this mechanism can be tailored to cure a large number of different genetic diseases. However, the main challenge is that HDR repair requires the delivery of CRISPR/Cas9, small guide RNA (sgRNA) and a donor DNA strand at the same time to a particular location. This requirement becomes particularly limiting for in vivo applications because ensuring co-delivery of multiple large molecules to the same targeted location is currently not feasible. For example, the Cas9 enzyme sequence and guide RNA complex is too large to fit into AAVs.

Thus, there is a need for effective non-viral delivery systems, including gene delivery systems. The current state-of-the-art non-viral gene delivery systems, such as liposomes, have many drawbacks such as poor biocompatibility and the inability to easily engineer or functionalize them. Additional concerns are that such non-viral gene delivery systems are easily degraded by various enzymes as they pass through intracellular or intercellular compartments, and these systems have not been able to package multiple large payloads.

### SUMMARY

In some aspects, the disclosure provides block copolymer comprising:
a first block homopolymer of monomer units, wherein each monomer unit is represented by formula I:

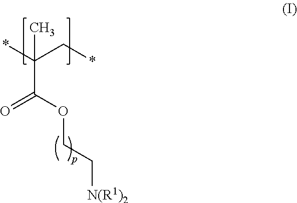

or a salt thereof, wherein:
   p is 0 or an integer selected from 1-3,
   each R¹ is individually selected from H or C₁-C₆ alkyl; and
   wherein each * individually represents a point of covalent attachment to the rest of the block copolymer; and
   a second block of either:
   (i) a homopolymer second block of monomer units, wherein each monomer unit is represented by formula II:

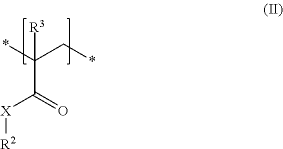

or a salt thereof, wherein:
   X is —O— or —NH—;
   R² is H or C₁-C₆ alkyl; and
   R³ is C₁-C₃ alkyl; or
   (ii) a copolymer second block comprising a copolymer of two or more monomer units each individually represented by formula II:

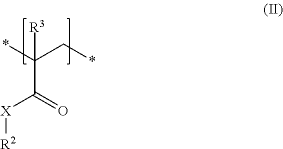

or a salt thereof, wherein:
   X is —O— or —NR²—
   each R² is H, C₁-C₆ alkyl, or polyethylene glycol, wherein each hydrogen in C₁-C₆ alkyl is optionally substituted by amino; and
   R³ is C₁-C₃ alkyl;
   wherein each * individually represents a point of covalent attachment to the rest of the block copolymer; and
wherein the first block has a molecular weight (Mw) of at least 9,000 Da.

In another aspect, the disclosure provides a block copolymer that comprises a first block and a second block, wherein:
the first block comprises a homopolymer of poly dimethylaminoethyl methacrylate (DMAEMA) or poly aminoethyl methacrylate (AEMA), and
the second block comprises either:
   (a) a homopolymer of polymethylmethacrylate (MMA) or polybutylmethacrylate (BMA); or
   (b) a copolymer of at least two of DMAEMA, MMA, BMA, polyethyleneglycol methacrylate (PEGMA), methylacrylic acid (MAA), and ethylacrylic acid (EAA),
wherein the first block has a molecular weight (Mw) of at least 9,000 Da.

In another aspect, the disclosure provides a polymer nanoparticle comprising a block copolymer according to the present disclosure, wherein the nanoparticle has a diameter of about 20 nm to about 2000 nm, for example about 50 nm to about 500 nm.

In another aspect, the disclosure provides a composition comprising a polymer nanoparticle according to the present disclosure and a nucleic acid complexed to the polymer nanoparticle.

In another aspect, the disclosure provides a method of treating a disease in a patient in need thereof, the method comprising administering a therapeutically effective amount of a composition according to the present disclosure.

In another aspect, the disclosure provides a method of treating a disease in a patient in need thereof, the method comprising delivering a therapeutically effective amount of a nucleic acid to the central nervous system (e.g., the brain or the sciatic nerve) of a patient using the polymer nanoparticle according to the present disclosure.

Additional embodiments, features, and advantages of the disclosure will be apparent from the following detailed description and through practice of the disclosure. The compounds of the present disclosure can be described as embodiments in any of the following enumerated clauses. It will be understood that any of the embodiments described herein can be used in connection with any other embodiments described herein to the extent that the embodiments do not contradict one another.

### BRIEF DESCRIPTION OF THE DRAWINGS

FIG. 1 shows normalized DNA barcode counts found in mouse sciatic nerve tissue.

FIG. 2A depicts Block 1 polymer characterization, and shows a bar graph of the percent monomer conversion as measured by proton nuclear magnetic resonance (NMR).

FIG. 2B depicts Block 1 polymer characterization, and shows a bar graph of molecular weight as measured by a conventional polystyrene standard curve gel permeation chromatography (GPC) method.

FIG. 3A is a box scatter plot that shows the PDI of the nanoparticles on the y-axis and the nanoparticle diameter on the x-axis of the first library including a first block having a DMAEMA homopolymer and a second block having an MMA homopolymer that was prepared. Reference lines for PDI values of 0.3, which is the PDI gold standard for PNP measurements using Dynamic Light Scattering, and 0.571, which is the instrument measurement limit.

FIG. 3B is a box scatter plot that shows the PDI of the nanoparticles on the y-axis and the nanoparticle diameter on the x-axis of the second library including a first block having a DMAEMA homopolymer and a second block having a DMAEMA, MMA, and MAA copolymer that was prepared. Reference lines for PDI values of 0.3, which is the PDI gold standard for PNP measurements using Dynamic Light Scattering, and 0.571, which is the instrument measurement limit.

FIG. 3C is a box scatter plot that shows the PDI of the nanoparticles on the y-axis and the nanoparticle diameter on the x-axis of the third library including a first block having an AEMA homopolymer and a second block having an MMA homopolymer that was prepared. Reference lines for PDI values of 0.3, which is the PDI gold standard for PNP measurements using Dynamic Light Scattering, and 0.571, which is the instrument measurement limit.

FIG. 3D is a box scatter plot that shows the PDI of the nanoparticles on the y-axis and the nanoparticle diameter on the x-axis of the fourth library including a first block having a DMAEMA homopolymer and a second block having a DMAEMA, MMA, and EAA copolymer that was prepared. Reference lines for PDI values of 0.3, which is the PDI gold standard for PNP measurements using Dynamic Light Scattering, and 0.571, which is the instrument measurement limit.

FIG. 3E is a box scatter plot that shows the PDI of the nanoparticles on the y-axis and the nanoparticle diameter on the x-axis of the fifth library including a first block having a DMAEMA homopolymer and a second block having an MMA and MAA copolymer that was prepared. Reference lines for PDI values of 0.3, which is the PDI gold standard for PNP measurements using Dynamic Light Scattering, and 0.571, which is the instrument measurement limit.

FIG. 3F is a box scatter plot that shows the PDI of the nanoparticles on the y-axis and the nanoparticle diameter on the x-axis of the sixth library including a first block having a DMAEMA homopolymer and a second block having an MMA and PEGMA copolymer that was prepared. Reference lines for PDI values of 0.3, which is the PDI gold standard for PNP measurements using Dynamic Light Scattering, and 0.571, which is the instrument measurement limit.

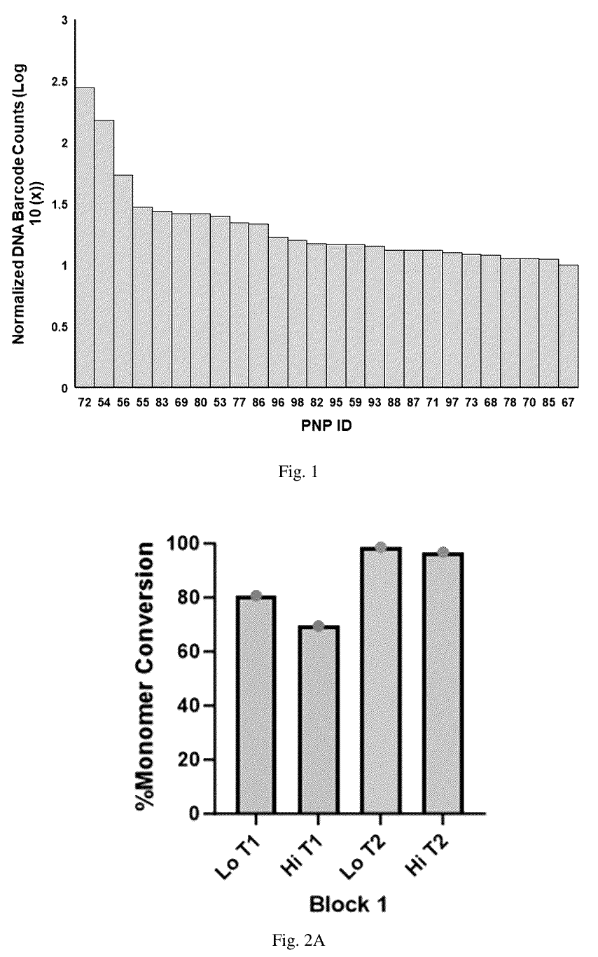

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended clauses.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein, the singular forms “a,” “an,” and “the” include plural referents unless the context clearly dictates otherwise. It is further noted that the clauses may be drafted to exclude any optional element. As such, this statement is intended to serve as antecedent basis for use of such exclusive terminology as “solely,” “only” and the like in connection with the recitation of clause elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” may be an approximation of ±10%, ±5%, or ±1%. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2014 (ACD/Labs) or ChemBioDraw Ultra 13.0 (Perkin Elmer).

As used herein and in connection with chemical structures depicting the various embodiments described herein, “*”, “**” and “ [symbol] ”, each represent a point of covalent attachment of the chemical group or chemical structure in which the identifier is shown to an adjacent chemical group or chemical structure. For example, in a hypothetical chemical structure A-B, where A and B are joined by a covalent bond, in some embodiments, the portion of A-B defined by the group or chemical structure A can be represented by“A—*”, “A—**”, or

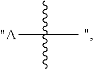

where each of and “—*”, “—**”, and

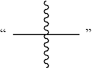

represents a bond to A and the point of covalent bond attachment to B. Alternatively, in some embodiments, the portion of A-B defined by the group or chemical structure B can be represented by “*—B”, “**—B”, or

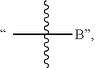

where each of “—*”, “—**”, and

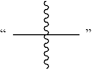

represents a bond to B and the point of covalent bond attachment to A.

As used herein, “molecular weight” refers to the weight average molecular weight (Mw) determined by a conventional polystyrene standard curve gel permeation chromatography method (hereinafter referred to as GPC) using multiple narrow distribution polystyrene standard samples relevant to the present invention and preferably between 2 kDa to 1,000 kDa.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### CHEMICAL DEFINITIONS

The term “alkyl” refers to a straight- or branched-chain monovalent hydrocarbon group. The term “alkylene” refers to a straight- or branched-chain divalent hydrocarbon group. In some embodiments, it can be advantageous to limit the number of atoms in an “alkyl” or “alkylene” to a specific range of atoms, such as C₁-C₂₀ alkyl or C₁-C₂₀ alkylene, C₁-C₁₂ alkyl or C₁-C₁₂ alkylene, or C₁-C₆ alkyl or C₁-C₆ alkylene. Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. Examples of alkylene groups include methylene (—CH₂—), ethylene ((—CH₂—)₂), n-propylene ((—CH₂—)₃), iso-propylene ((—C(H)(CH₃)CH₂—)), n-butylene ((—CH₂—)₄), and the like. It will be appreciated that an alkyl or alkylene group can be unsubstituted or substituted as described herein. An alkyl or alkylene group can be substituted with any of the substituents in the various embodiments described herein, including one or more of such substituents. Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀ alkyl indicates a hydrogen where the group is in a terminal position, a bond if internal. A C₁₋₆ alkyl group, for example, contains from one to six carbon atoms in the chain.

The term “alkenyl” refers to a straight- or branched-chain mono-valent hydrocarbon group having one or more double bonds. In some embodiments, it can be advantageous to limit the number of atoms in an “alkenyl” to a specific range of atoms, such as C₂-C₂₀ alkenyl, C₂-C₁₂ alkenyl, or C₂-C₆ alkenyl. Examples of alkenyl groups include ethenyl (or vinyl), allyl, and but-3-en-1-yl. Included within this term are cis and trans isomers and mixtures thereof. It will be appreciated that an alkenyl can be unsubstituted or substituted as described herein. An alkenyl group can be substituted with any of the substituents in the various embodiments described herein, including one or more of such substituents.

The term “amide”, as used herein, refers to a group

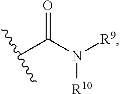

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

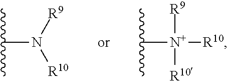

wherein R⁹, R¹⁰, and R^{10′}, each independently represent a hydrogen or a hydrocarbyl group, or R⁹ and R¹⁰ taken together with the N atom to which they are attached complete a heterocycle having from 4 to 8 atoms in the ring structure.

The term “alkylamino”, as used herein, refers to an amino group substituted with at least one alkyl group.

The term “carboxy”, as used herein, refers to a group represented by the formula —CO₂H.

The term “ester”, as used herein, refers to a group —C(O)OR⁸ wherein R⁸ represents a hydrocarbyl group.

The terms “halo” and “halogen” as used herein means halogen and includes chloro, fluoro, bromo, and iodo.

The term “heteroatom” as used herein means an atom of any element other than carbon or hydrogen. Preferred heteroatoms are nitrogen, oxygen, and sulfur.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “substituted” means that the specified group or moiety bears one or more substituents. The term “unsubstituted” means that the specified group bears no substituents. Where the term “substituted” is used to describe a structural system, the substitution is meant to occur at any valency-allowed position on the system. In some embodiments, “substituted” means that the specified group or moiety bears one, two, or three substituents. In other embodiments, “substituted” means that the specified group or moiety bears one or two substituents. In still other embodiments, “substituted” means the specified group or moiety bears one substituent.

Any formula depicted herein is intended to represent a compound of that structural formula as well as certain variations or forms. For example, a formula given herein is intended to include a racemic form, or one or more enantiomeric, diastereomeric, or geometric isomers, or a mixture thereof. Additionally, any formula given herein is intended to refer also to a hydrate, solvate, or polymorph of such a compound, or a mixture thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

The nomenclature “(ATOM)ᵢ₋(ATOM)ⱼ” with j>i, when applied herein to a class of substituents, is meant to refer to embodiments of this disclosure for which each and every one of the number of atom members, from i to j including i and j, is independently realized. By way of example, the term C₁-C₃ refers independently to embodiments that have one carbon member (C₁), embodiments that have two carbon members (C₂), and embodiments that have three carbon members (C₃).

“Acceptable salt” or “salt” is used herein to refer to an acid addition salt or a basic addition salt which is suitable for or compatible with the compounds or a desired treatment.

The disclosure also includes acceptable salts (e.g., pharmaceutically acceptable salts) of the block copolymers described herein, preferably of those described above and of the specific compounds exemplified herein, and pharmaceutical compositions comprising such salts, and methods of using such salts.

A “pharmaceutically acceptable salt” is intended to mean a salt of a free acid or base of a compound represented herein that is non-toxic, biologically tolerable, or otherwise biologically suitable for administration to the subject. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

It will be understood that the chemical entities described herein, can exist as a salt of a free acid or base of a compound represented herein and an inorganic or organic counter ion. Illustratively, the salt can be formed during the manufacture of the compound described herein (e.g., a salt or a pharmaceutically acceptable salt) or a compound described herein can be substituted to provide a salt for further manufacture, formulation, or administration reasons. As illustrated herein, certain compounds include a “W\textcircled{−}),” wherein “W\textcircled{−}” is an inorganic counter ion (e.g., an inorganic anion) or an organic counter ion (e.g., an organic anion). In certain embodiments, W\textcircled{−} is an anion that is complexed with a cation of a compound of the disclosure to form a pharmaceutically acceptable salt.

The term “inorganic counter ion” represents an inorganic ion that accompanies an ionic species in order to maintain electric neutrality. An inorganic counter ion may represent an anion or cation. An inorganic counterion may accompany a free acid or base of a compound represented herein. An inorganic ion may form by a reaction of an inorganic base or inorganic acid and a compound described herein that possesses a sufficiently acidic group, a sufficiently basic group, both types of functional groups, or more than one of each type.

The term “organic counter ion” represents an organic counter ion that accompanies an ionic species in order to maintain electric neutrality. The organic ion may represent an anion or cation. An organic counter ion may accompany a free acid or base of a compound represented herein. An organic ion may form by a reaction of an organic base or organic acid and a compound described herein that possesses a sufficiently acidic group, a sufficiently basic group, both types of functional groups, or more than one of each type.

The term “acceptable acid addition salt” as used herein means any non-toxic organic or inorganic salt of any base compounds of the present disclosure. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of the present disclosure are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of the present disclosure for laboratory use, or for subsequent conversion to a pharmaceutically acceptable acid addition salt.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a block copolymer that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit disease progression or reverse disease may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a compound or a composition which reduces the frequency of, or delays the onset of, symptoms of a medical condition in a subject relative to a subject which does not receive the compound or composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance (e.g., a compound, a composition, or an agent) to a subject can be carried out using one of a variety of methods known to those skilled in the art. For example, a substance can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A substance (e.g., a compound, a composition, or agent) can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance (e.g., a compound, a composition, or an agent) to a subject will also depend, for example, on the age and/or the physical condition of the subject and the chemical and biological properties of the compound or agent (e.g., solubility, digestibility, bioavailability, stability and toxicity). In some embodiments, a substance (e.g., a compound, a composition, or an agent) is administered orally, e.g., to a subject by ingestion. In some embodiments, the orally administered substance is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present disclosure can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O— alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “modulate” as used herein includes the inhibition or suppression of a function or activity (such as cell proliferation) as well as the enhancement of a function or activity.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

As used herein and in connection with chemical structures depicting the various embodiments described herein, a polymer may be arranged in any linear or branched configuration. For example, a polymer comprising one or more monomer units may be arranged as a homopolymer, a block copolymer, a statistical copolymer, a random copolymer, or an alternate copolymer of different monomers linked together in an alternating fashion. In one embodiment, the polymers described herein may include repeating blocks, such as in a block copolymer, of units selected from formula I and formula II.

The term “block copolymer” is known in the art, and a representative definition is that a block copolymer is a polymer comprising molecules in which there is a linear arrangement of blocks, for example a block A linearly connected to a block B, where each of the blocks (e.g., block A and block B) comprises units derived from a characteristic species or combination of species of monomer such that at least one difference exists between the species of monomer(s) of the different blocks (e.g., the monomer species or combination of species of block A is different than the monomer species or combination of species of block B.

The term “statistical copolymer” is known in the art, and a representative definition is that a statistical copolymer can be a copolymer composed of monomers that form a sequence based on a statistical rule (e.g., Markovian statistics).

The term “random copolymer” is known in the art, and a representative definition is that a random copolymer describes a copolymer where the probability of finding a given type monomer residue at a particular point in the chain is equal to the mole fraction of that monomer residue in the chain and is independent of the neighboring units in the chain.

The term “alternate copolymer” is known in the art, and a representative definition is that an alternate copolymer describes a copolymer of monomers sequentially linked in a uniform pattern.

“Nucleotide” as used herein is a molecule that contains a base moiety, a sugar moiety, and a phosphate moiety. Nucleotides can be linked together through their phosphate moieties and sugar moieties creating an internucleoside linkage. The term “oligonucleotide” is sometimes used to refer to a molecule that contains two or more nucleotides linked together. The base moiety of a nucleotide can be adenine-9-yl (A), cytosine-1-yl (C), guanine-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide that contains some type of modification to the base, sugar, and/or phosphate moieties. Modifications to nucleotides are well known in the art and would include, for example, 5-methylcytosine (5-me-C), 5 hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but are linked together through a moiety other than a phosphate moiety. Nucleotide substitutes are able to conform to a double helix type structure when interacting with the appropriate target nucleic acid.

The term “polynucleotide,” as used herein, means a molecule including one or more nucleic acid subunits, or nucleotides, and can be used interchangeably with “nucleic acid” or “oligonucleotide”. A polynucleotide can include one or more nucleotides selected from adenosine (A), cytosine (C), guanine (G), thymine (T) and uracil (U), or variants thereof. A nucleotide can include a nucleoside and at least 1, 2, 3, 4, 5, 6, 7, 8, 9, 10, or more phosphate (PO₃) groups. A nucleotide can include a nucleobase, a five-carbon sugar (either ribose or deoxyribose), and one or more phosphate groups. Ribonucleotides are nucleotides in which the sugar is ribose. Polyribonucleotides or ribonucleic acids, or RNA, can refer to macromolecules that include multiple ribonucleotides that are polymerized via phosphodiester bonds. Deoxyribonucleotides are nucleotides in which the sugar is deoxyribose. As used herein, a polyribonucleotide sequence that recites thymine (T) is understood to represent uracil (U).

“Polydeoxyribonucleotides,” “deoxyribonucleic acids,” and “DNA” mean macromolecules that include multiple deoxyribonucleotides that are polymerized via phosphodiester bonds. “Polyribonucleotides,” “ribonucleic acids,” and “RNA” mean macromolecules that include multiple ribonucleotides that are polymerized via phosphodiester bonds. A nucleotide can be a nucleoside monophosphate or a nucleoside polyphosphate. A nucleotide means a deoxyribonucleoside polyphosphate, such as, e.g., a deoxyribonucleoside triphosphate (dNTP), which can be selected from deoxyadenosine triphosphate (dATP), deoxycytidine triphosphate (dCTP), deoxyguanosine triphosphate (dGTP), deoxyuridine triphosphate (dUTP) and deoxythymidine triphosphate (dTTP) dNTPs, and may include detectable tags, such as protein tags, luminescent tags or markers (e.g., fluorophores). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof).

The term “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini.

The term “amino acid sequence” refers to a series of two or more amino acids linked together via peptide bonds wherein the order of the amino acids linkages is designated by a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

The term “vector” or “construct” designates a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “operably linked to” refers to the functional relationship of a nucleic acid with another nucleic acid sequence. Promoters, enhancers, transcriptional and translational stop sites, and other signal sequences are examples of nucleic acid sequences that can operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

DNA and RNA can be synthesized naturally (e.g., by DNA replication or transcription of DNA or RNA, respectively). DNA and RNA can also be chemically synthesized. RNA can be post-transcriptionally modified. The terms “target mRNA” and “target transcript,” “target sequence” are synonymous as used herein.

As used herein, “target sequence” refers to a contiguous portion of the nucleotide sequence of an mRNA molecule formed during the transcription of a gene, including mRNA that is a product of RNA processing of a primary transcription product.

“Targeting” an oligomeric compound to a particular nucleic acid molecule, in the context of this invention, can be a multistep process. The process usually begins with the identification of a target nucleic acid whose function is to be modulated. This target nucleic acid may be, for example, a cellular gene (or mRNA transcribed from the gene) whose expression is associated with a particular disorder or disease state, or a nucleic acid molecule from an infectious agent.

The targeting process usually also includes determination of at least one target region, segment, or site within the target nucleic acid for the antisense interaction to occur such that the desired effect, e.g., modulation of expression, will result. Within the context of the present invention, the term “region” is defined as a portion of the target nucleic acid having at least one identifiable structure, function, or characteristic. Within regions of target nucleic acids are segments. “Segments” are defined as smaller or sub-portions of regions within a target nucleic acid. “Sites,” as used in the present invention, are defined as positions within a target nucleic acid.

The term “RNAi” as used herein refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but are not limited to, siRNAi, shRNAi, endogenous microRNA and artificial microRNA. For instance, it includes sequences previously identified as siRNA, regardless of the mechanism of down-stream processing of the RNA (i.e., although siRNAs are believed to have a specific method of in vivo processing resulting in the cleavage of mRNA, such sequences can be incorporated into the vectors in the context of the flanking sequences described herein).

The term “RNAi” and “RNA interfering” with respect to an agent of the invention, are used interchangeably herein. RNAi molecules are typically comprised of a sequence of nucleic acids or nucleic acid analogs, specific for a target gene. A nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA).

As used herein, “shRNA” or “small hairpin RNA” (also called stem loop) is a type of RNAi. shRNAs may be composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow. shRNAs functions as RNAi but are further defined in that shRNA species are double stranded hairpin-like structure for increased stability. These shRNAs, as well as other such agents described herein, can be contained in plasmids, retroviruses, and lentiviruses and expressed from, for example, the pol III U6 promoter, or another promoter.

### Representative Embodiments

This disclosure describes compositions of cationic polymers (e.g., diblock copolymers) which self-assemble in aqueous conditions to form polymer nanoparticles (PNPs). In illustrative embodiments, the PNPs can complex nucleic acids and deliver the complexed nucleic acids to a desired location. For example, the complexes can deliver the nucleic acids to the central nervous system (e.g., the sciatic nerve) or to brain tissue.

In certain embodiments, a block copolymer comprises a first block and a second block. The first block may comprise a homopolymer of poly dimethylaminoethyl methacrylate (DMAEMA) or poly aminoethyl methacrylate (AEMA). The second block may comprise a homopolymer or a copolymer such as a statistical copolymer. For example, the second block may comprise a homopolymer of polymethylmethacrylate (MMA). Alternatively, the second block may comprise a copolymer of at least two of DMAEMA, MMA, polyethyleneglycol methacrylate (PEGMA), methylacrylic acid (MAA), and ethylacrylic acid (EAA). In certain preferred embodiments, the block copolymer is a diblock copolymer.

In certain embodiments, a block copolymer comprises a first block homopolymer of monomer units, wherein each monomer is unit represented by formula I:

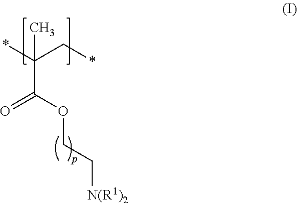

or a salt thereof, wherein:
p is 0 or an integer selected from 1-3,
each R¹ is individually selected from H or C₁-C₆ alkyl; and
wherein each * individually represents a point of covalent attachment to the rest of the block copolymer.

In certain embodiments, a block copolymer comprises a second block homopolymer of monomer units, wherein each monomer unit is represented by formula II:

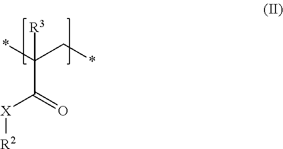

or a salt thereof, wherein:
   X is —O— or —NH—;
   R² is H or C₁-C₆ alkyl; and
R³ is C₁-C₃ alkyl,
wherein each * individually represents a point of covalent attachment to the rest of the block copolymer.

Alternatively, a block copolymer comprises second block copolymer comprising a copolymer of two or more monomer units each individually represented by formula II:

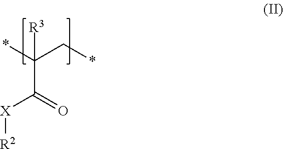

or a salt thereof, wherein:
   X is —O— or —NR²—;
   each R² is H, C₁-C₆ alkyl, or polyethylene glycol, wherein each hydrogen in C₁-C₆ alkyl is optionally substituted by amino; and
   R³ is C₁-C₃ alkyl;
wherein each * individually represents a point of covalent attachment to the rest of the block copolymer. For example, the copolymer can be a statistical copolymer.

In certain embodiments, p is 0. Alternatively, p may be a value greater than 0, for example a value selected from 1-3, and is preferably 1.

In certain embodiments, each R¹ is C₁-C₃ alkyl. For example each R¹ can be methyl or isopropyl, and is preferably methyl. In certain embodiments, each R¹ is H.

In certain embodiments, the second block comprises a homopolymer of a monomer unit represented by formula II. In certain embodiments, the second block comprises a copolymer, for example a statistical copolymer, comprising a first monomer unit represented by formula II and a second monomer unit represented by formula II.

In certain embodiments, for example those where the second block comprises a homopolymer, X is preferably —O—. In certain embodiments where the second block comprises a copolymer, X is —O— in a monomer unit, for example the first monomer unit, the second monomer unit, the third monomer unit, or a combination thereof.

In certain embodiments, R² is C₁-C₆ alkyl, preferably methyl or butyl. In certain embodiments, R² is C₁-C₆ alkyl, for example methyl. In certain embodiments where the second block comprises a copolymer, R² is C₁-C₆ alkyl (e.g., methyl) in a monomer unit, for example the first monomer unit, the second monomer unit, the third monomer unit, or a combination thereof, and may optionally be substituted by amino (i.e., —N(C₁-C₆ alkyl)₂ such as dimethylamino). In certain embodiments where the second block comprises a copolymer, R² is C₁-C₆ alkyl (e.g., methyl or butyl) in a monomer unit, for example the first monomer unit, the second monomer unit, the third monomer unit, or a combination thereof, and may optionally be substituted by amino (i.e., —N(C₁-C₆ alkyl)₂ such as dimethylamino). For example, R² may be dimethylaminoethyl. In certain embodiments where the second block comprises a copolymer, R² is H in a monomer unit, for example the first monomer unit, the second monomer unit, the third monomer unit, or a combination thereof. In certain embodiments where the second block comprises a copolymer, R² is a polyethylene glycol (e.g., a PEG with a (Mn) of about 100 to about 500 Da, such as about 360 Da) in a monomer unit, for example the first monomer unit, the second monomer unit, the third monomer unit, or a combination thereof.

In certain embodiments, R³ is methyl. In certain embodiments, R³ is ethyl.

In some embodiments, the first block can be prepared from one or more monomer units and have a molecular weight (M_{w}) in the range of about 9 kDa to about 100 kDa, about 15 kDa to about 100 kDa, about 20 kDa to about 100 kDa, about 20 kDa to about 80 kDa, about 20 kDa to about 70 kDa, or about 20 kDa to about 60 kDa. In some embodiments, the first block can be prepared from one or more monomer units and have a molecular weight (M_{w}) in the range of about 25 kDa to about 100 kDa, or about 25 kDa to about 80 kDa, or about 25 kDa to about 70 kDa, or about 25 kDa to about 60 kDa.

In some embodiments, the second block has a degree of polymerization of at least 50. For example, in some embodiments, the degree of polymerization is about 50 to about 800, about 100 to about 700, about 100 to about 800, about 200 to about 800, or about 200 to about 700. In some embodiments, the second block has a degree of polymerization of at least 200, for example about 200 to about 800 or about 200 to about 350. In some embodiments, the second block has a degree of polymerization of at least 250. In some embodiments, the second block has a degree of polymerization of about 250 to about 800, about 350 to about 800, or about 350 to about 700. In some embodiments, the second block has a degree of polymerization of at least 250 for example about 250 to about 800. In some embodiments,

In some embodiments, the second block is less than about 80 mol % of the block copolymer. In some embodiments, the second block is less than about 55 mol % or less than about 30 mol % of the block copolymer. In some embodiments, the second block is about 70 mol % to about 80 mol %, about 45 mol % to about 55 mol %, or about 20 mol % to about 30 mol % of the block copolymer.

In some embodiments, block copolymers as prepared herein can be described by the following structure:

CTACap-[first block 1]ₘ-[second block]ₙ-CTACap

where each CTACap is a capping unit derived from the chain transfer agent(s) used in the process for preparing the RAFT copolymer. The CTA used for preparing each of the first block and the second block can be the same or different. In some embodiments, the CTA used to prepare each the first block and the second block is the same (e.g., macroCTA). In some embodiments, the CTA used to prepare each of the first block and the second block is different. In some embodiments, the CTA used to prepare one or both of the first block and the second block comprises a functional group for the covalent attachment of a biomolecule, drug, or label to the block copolymer. In some embodiments, the covalent attachment can be via an ester or an amide bond. In some embodiments, the covalent attachment can be via EDC-NHS chemistry.

In some embodiments, the first block comprises a cap of formula:

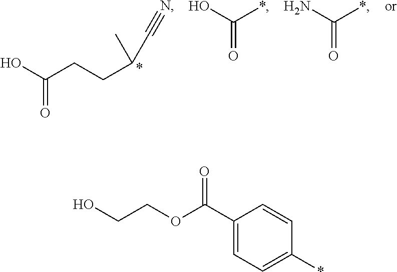

or a salt thereof, wherein * represents a point of covalent attachment to the first block. In certain preferred embodiments, the cap is

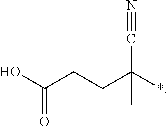

In some embodiments, the second capping unit is of formula

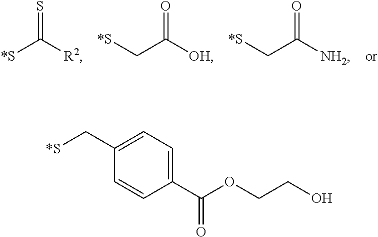

or a salt thereof, wherein * represents a point of covalent attachment to the second block, and R² is —SC₂-C₁₂ alkyl or C₆H₅, and is preferably

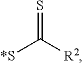

such as

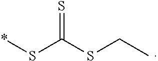 [SMILES: *SC(=S)SCC]

In certain preferred embodiments, the first block of the block copolymer comprises 2-aminoethylmethacrylamide (AEMA) or poly dimethylaminoethyl methacrylate (DMAEMA) as the first block. The second block of the block copolymer comprises either (a) a homopolymer of polymethylmethacrylate (MMA) or (b) a copolymer of at least two of DMAEMA, MMA, polyethyleneglycol methacrylate (PEGMA), methylacrylic acid (MAA), and ethylacrylic acid (EAA). The block copolymer is preferably a diblock copolymer.

In certain preferred embodiments that biodistribute to the brain, the block copolymer comprises a first block that comprises DMAEMA. The second block comprises a homopolymer of MMA, or alternatively comprises (i) a copolymer of DMAEMA (10-20%), MMA (50-70%), EAA (20-30%); (ii) DMAEMA (15-25%), MMA (55-70%, and MAA (5-30%), (iii) MMA (70%) and PEGMA (30%); (iv) MMA (70%) and MAA (30%). The second block preferably comprises a homopolymer of MMA, or alternatively comprises (i) a copolymer of DMAEMA (10-20%), MMA (50-80%), EAA (10-30%); (ii) DMAEMA (15-25%), MMA (55-70%, and MAA (5-30%), (iii) MMA (70%) and PEGMA (30%); (iv) MMA (70%) and MAA (30%). In certain preferred embodiments, the first block is a DMAEMA homopolymer and has a molecular weight of about 15 kDa to about 45 kDa, the second block is a homopolymer of BMA or MMA, wherein the degree fo polymerization in the second block is about 350 to about 575, and the PNP has a diameter of about 220 nm to about 350 nm.

In certain preferred embodiments that biodistribute to the sciatic nerve, the block copolymer comprises a first block that comprises DMAEMA. The second block preferably comprises a homopolymer of MMA, or alternatively comprises (i) a copolymer of MMA (70-80%) and PEGMA (20-30%); or (ii) DMAEMA (10-20%), MMA (50-80%), or EAA (10-30%). In certain preferred embodiments, the first block is a DMAEMA homopolymer and has a molecular weight of about 30 kDa to about 45 kDa, the second block is a homopolymer of BMA or MMA, wherein the degree fo polymerization in the second block is about 450 to about 575, and the PNP has a diameter of about 220 nm to about 350 nm.

In some embodiments, a polymer nanoparticle comprises a block copolymer according to the present disclosure. In some embodiments, the block copolymer self-assembles into the nanoparticle.

In some embodiments, a diameter (e.g., a hydrodynamic diameter) of the nanoparticle is about 20 nm to about 2,000 nm, about 150 nm to about 500 nm, about 200 nm to about 500 nm, about 280 nm to about 390 nm, or about 300 nm to about 360 nm, as measured by DLS. In some embodiments, a diameter (e.g., a hydrodynamic diameter) of the nanoparticle is about 175 nm to about 350 nm, for example is about 200 nm to about 320 nm, as measured by DLS. In certain embodiments, the nanoparticle has a diameter of about 75 nm to about 700 nm, for example about 75 nm to about 250 nm. In some embodiments, the nanoparticle has a diameter of about 150 nm to about 700 nm. In some embodiments, the nanoparticle has a diameter of about 125 nm to about 400 nm. In some embodiments, the nanoparticle has a diameter of about 200 nm to about 500 nm, for example about 230 nm to about 320 nm.

In certain embodiments that biodistribute to the sciatic nerve, the diameter of the nanoparticle is about 15 nm to about 2,000 nm, about 150 nm to about 1,000 nm, about 250 nm to about 700 nm, or about 20 nm to about 500 nm, as measured by DLS. In certain embodiments, nanoparticles that biodistributed to the brain have a diameter of about 280 nm to about 390 nm. In certain embodiments, nanoparticles that biodistributed to the sciatic nerve has a diameter of about 300 nm to about 360 nm. In certain embodiments, nanoparticles that biodistributed to the sciatic nerve has a diameter of about 50 nm to about 275 nm.

In certain embodiments that biodistribute to the brain, the diameter of the nanoparticle is about 20 nm to about 1,400 nm, about 20 nm to about 950 nm, about 150 nm to about 950 nm, about 150 nm to about 600 nm, or about 20 nm to about 500 nm, as measured by DLS. In certain embodiments, nanoparticles that biodistributed to the brain have a diameter of about 220 nm to about 350 nm.

In certain embodiments, the nanoparticle preferably has a polydispersity index (PDI) of lower than about 0.3. For example, the nanoparticle can have a PDI of about 0.05 to about 3, about 0.1 to about 0.3, about 0.1 to about 0.2, or about 0.2 to about 0.3.

In some embodiments, a composition comprises a polymer nanoparticle as described herein (and a nucleic acid (sometimes called a payload) complexed to the polymer nanoparticle. For example, the nucleic acid may be complexed to the nanoparticle through electrostatic interactions. In certain preferred embodiments, the polymer nanoparticle can serve as a transfection agent to deliver a nucleic acid to a cell.

In certain embodiments, the nucleic acid is an RNA (e.g., an mRNA, a microRNA, antisense oligonucleotides, or an siRNA) or a DNA (e.g., an ssDNA, a dsDNA, a complimentary coding DNA (cDNA), or a DNA plasmid (pDNA)). In certain embodiments, the nucleic acid is an mRNA. In certain embodiments, the nucleic acid is a circular RNA. In certain embodiments, the nucleic acid is a DNA plasmid (pDNA).

For example, the transfection efficiency is at least about 0.1%, at least about 0.3%, at least about 0.5%, at least about 0.7%, at least about 0.9%, or at least about 1%. As another example, the transfection efficiency is at least about 0.1%, at least about 0.3%, at least about 0.5%, at least about 0.7%, at least about 0.9%, or at least about 1% into neuronal cells such as Schwann cells. In some embodiments, the transfection efficiency (e.g., at a PNP:DNA ratio of about 10:1, about 15:1, or about 30:1 by weight) is at least about 1%, at least about 3%, or about least about 9% in cells (e.g., neuronal cells such as Schwann cells). For example, the transfection efficiency (e.g., at a PNP:DNA ratio of about 10:1, about 15:1, or about 30:1 by mass) may be about 1% to about 20%, about 1% to about 18%, about 1% to about 12%, about 1% to about 10%, about 1% to about 6%, about 1% to about 5%, about 2% to about 18%, about 2% to about 16%, about 5% to about 16%, or about 10% to about 20% in cells (e.g., neuronal cells such as Schwann cells). For example, the transfection efficiency may be about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% in cells (e.g., neuronal cells such as Schwann cells).

In some embodiments, the nucleic acid is at least 5 kbp or at least about 10 kbp. In certain embodiments, the nucleic acid is about 10 kbp to about 15 kbp.

In certain embodiments, the ratio of nucleic acid to nanoparticle is about 5:1 to about 60:1 by weight. For example, the ratio of nanoparticle to nucleic acid (e.g., DNA such as pDNA) (PNP:DNA) may be about 10:1, about 15:1, or about 30:1 by weight. The polymer nanoparticles described herein are capable of interacting with (e.g., encapsulating) nucleotides. The polymer nanoparticles described herein are capable of interacting with (e.g., encapsulating) nucleotide plasmids. In some embodiments, the encapsulation efficiency is greater than about 70%, greater than about 75%, greater than about 80%, greater than about 85%, greater than about 90%, greater than about 95%, or greater than about 97%. For example, the encapsulation efficiency for a nucleotide (e.g., for DNA such as pDNA at a PNP:DNA ratio of 10:1, 15:1, or 30:1) may be about 70% to about 100%, about 75% to about 100%, about 80% to about 100%, about 90% to about 100%, or about 95% to about 100%. For example, the encapsulation efficiency for a nucleotide (e.g., for DNA such as pDNA at a PNP:DNA ratio of 10:1, 15:1, or 30:1) may be about 70%, about 75%, about 80%, about 85%, about 90%, about 95%, or about 100%.

In some embodiments, the polymer nanoparticles have low toxicity to cells. For example, in some embodiments, the cell viability after transfection of cells (e.g., neuronal cells such as Schwann cells) is at least about 70%, at least about 75%, at least about 80%, at least about 85%, at least about 90%, or at least about 95%. For example, in some embodiments, the cell viability after transfection of cells (e.g., neuronal cells such as Schwann cells using a nucleotide (e.g., for DNA such as pDNA at a PNP:DNA ratio of 10:1, 15:1, or 30:1) is at least about 70%, at least about 75%, at least about 80%, at least about 85%, at least about 90%, or at least about 95%. For example, the cell viability after transfection of cells (e.g., neuronal cells such as Schwann cells using a nucleotide (e.g., for DNA such as pDNA at a PNP:DNA ratio of 10:1 or 15:1) is about 70% to about 100%, about 70% to about 99%, about 75% to about 99%, about 80% to about 99%, about 85% to about 99%, about 90% to about 99%, or about 95% to about 99%.

In some embodiments, the nanoparticle comprises a barcode construct covalently attached to the nanoparticle, as described in U.S. Patent Application Publication No. 2022/033309, the entirety of which is hereby incorporated by reference. As described in U.S. Patent Application Publication No. 2022/033309, the presence of the barcode construct can allow for identification of PNPs of interest, for example by determining the presence of a PNP in particular tissue or by performance in an assay. It should be understood that although certain examples in this application are performed on PNPs that include the barcode, similar performance would be expected by PNPs that lack the barcode.

The compositions described herein can be used for treating disease. For example, the polymer nanoparticles may be able to deliver a nucleic acid (i.e., a payload) that provides a therapeutic benefit to a patient. In certain embodiments, the polymer nanoparticle is able to deliver the nucleic acid to cells, such as Schwann cells. In certain embodiments, the nanoparticles deliver nucleic acids to the brain or the central nervous system. For example, the nanoparticles described herein may deliver a nucleic acid to the sciatic nerve.

The nucleic acids delivered by the polymer nanoparticle may be useful in treating diseases such as those that affect the central nervous system (e.g., the brain or the sciatic nerve), but may be applied for the delivery of therapeutically cargo for other diseases.

In one embodiment a composition comprising a polymer nanoparticle (e.g., a polymer nanoparticle derived from a controlled living/radical polymerization process, such as RAFT polymer) associated with a nucleic acid construct is provided. In another embodiment, a method of treating a patient with a disease is provided comprising administering to the patient the polymer nanoparticle.

In some embodiments, a method of treating a patient with a disease is provided, comprising administering to the patient the polymer nanoparticle identified in the in vivo screening method, wherein the polymer nanoparticle further comprises a drug payload, such as a polynucleotide or a protein payload, or a small molecule therapeutic or luminescent molecule payload, and treating the disease in the patient.

In various embodiments, any suitable route for administration of the library of polymer nanoparticles associated with nucleic acid constructs for the method of in vivo screening for the polymer nanoparticle associated with a nucleic acid construct, or for the method of treatment can be used including parenteral administration. Suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. In one embodiment, means for parenteral administration include needle (including microneedle) injectors, needle-free injectors and infusion techniques. In other embodiments, oral or pulmonary routes of administration can be used.

In various embodiments, cell or tissue samples may be analyzed for the presence of the polymer nanoparticle associated with the nucleic acid constructs described herein. The samples can be any tissue, cell, or fluid sample from an animal, for example, selected from the group consisting of urine, nasal secretions, nasal washes, inner ear fluids, bronchial lavages, bronchial washes, alveolar lavages, spinal fluid, bone marrow aspirates, sputum, pleural fluids, synovial fluids, pericardial fluids, peritoneal fluids, saliva, tears, gastric secretions, stool, reproductive tract secretions, lymph fluid, whole blood, serum, plasma, or any tissue or cell sample from an animal. Exemplary tissue or cell samples include brain tissue or cells, muscle tissue or cells, skin tissue or cells, heart tissue or cells, kidney tissue or cells, stomach tissue or cells, liver tissue or cells, urinary tract tissue or cells, gastrointestinal tract tissue or cells, head or neck tissue or cells, lung tissue or cells, reproductive tract tissue or cells, pancreatic tissue or cells, or any other tissue or cell type from an animal.

In one illustrative aspect for removing cells or tissues from the animal and isolating the nucleic acid constructs from the cells or tissues of the animal, the nucleic acid constructs are removed from cells or tissues of the animal. In various embodiments, nucleic acid constructs (e.g., DNA or RNA) obtained from the tissues or cells of the animal can be removed by rupturing the cells and isolating the nucleic acid constructs from the lysate. Techniques for rupturing cells and for isolation of nucleic acids are well-known in the art, and removal techniques include homogenization, such as by using a bead-beating technique. In other embodiments, the nucleic acid constructs may be isolated by rupturing cells using a detergent or a solvent, such as phenol-chloroform. In another aspect, the nucleic acid constructs may be separated from the lysate by physical methods including, but not limited to, centrifugation, dialysis, diafiltration, filtration, size exclusion, pressure techniques, digestion of proteins with Proteinase K, or by using a substance with an affinity for nucleic acids such as, for example, beads that bind nucleic acids.

In various embodiments, payloads may be combined with the polymer nanoparticles compositions using any or all of covalent bonds, electrostatic interactions, and ligand affinity interactions. In one aspect, covalent bonding methods include the use of EDC/NHS to form stable amide bonds between the payload and the polymer nanoparticles for improved stability (both “on the shelf” and in vivo), ease of separation and extraction, and sensitive detection. In another illustrative aspect, electrostatic bonding methods include the use of cationic polymer nanoparticles that electrostatically complex with the payload. In another embodiment, ligand affinity bonding includes the use of ligands such as avidin and biotin, both covalently bonded to the polymer nanoparticles and the payload via EDC/NHS chemistry to yield the stable combination of the payload and the polymer nanoparticles.

It will be appreciated that RAFT polymerization is generally known in the art. Suitable reagents, monomers, and conditions for RAFT polymerization previously investigated can be used in the copolymers, methods, and compositions described herein, such as those described in U.S. Pat. Nos. 9,006,193, 9,464,300, and 9,476,063, the disclosures of each of which are incorporated by reference in their entirety.

Chain transfer agents (CTAs) useful in connection with the present disclosure are known in the art. The identity of the CTA is not particularly limited. It will be appreciated that chain transfers steps that form the basis of RAFT polymerization involve a reversible transfer of a functional chain end-group (typically a thiocarbonylthio group, Z—C(═S)S—R) between chains and the propagating radicals. The overall process is comprised of the insertion of monomers between the R— and Z—C(═S)S-groups of a RAFT agent (CTA), which form the α and ω end-group of the majority of the resulting polymeric chains. Suitable CTAs for use in connection with the present disclosure include but are not limited to trithiocarbonates (Z═S-alkyl), dithiobenzoates (Z═Ph), dithiocarbamate (Z═N-alkyl), xanthates (Z═O-alkyl), and the like. (See, Sébastien Perrier, Macromolecules 2017 50 (19), 7433-7447). In some embodiments, RAFT copolymerization may be achieved using chain transfer agents (CTAs) containing one or more terminal carboxyl groups in order to obtain carboxy terminated polymers with ends available for bonding to the payload via the methods described above. In this embodiment, when the resulting mono or di-carboxy terminated polymer is dispersed in a low pH (e.g., a pH of less than 6) buffer, both ends of the polymer are exposed and available for labeling via EDC/NHS chemistry. In this embodiment, when the polymer is transferred to a physiological pH (˜pH 7), the core blocks self-assemble, encapsulating the payload in the hydrophobic core, to be released and exposed upon acidification in the endosomal compartment of a cell. In some embodiments, the first or second chain transfer agent can be selected from the group consisting of bis(carboxymethyl)trithiocarbonate, bis(2-amino-2-oxoethyl) trithiocarbonate, bis[4-(2-hydroxyethoxycarbonyl)benzyl]trithiocarbonate, 4-cyano-4-(ethylsulfanylthiocarbonyl) sulfanylvpentanoic acid, 4-cyano-4-((phenylcarbonothioyl)thio)pentanoic acid, and 4-cyano-4-[(dodecylsulfanylthiocarbonyl)sulfanyl]pentanoic acid, 4-cyano-4-(thiobenzoylthio)pentanoic acid, 2-cyano-2-propyl benzodithioate, cyanomethyl methyl(phenyl)carbamodithioate, 2-cyano-2-propyl dodecyl trithiocarbonate, 2-(dodecylthiocarbonothioylthio)-2-methylpropionic acid, cyanomethyl dodecyl trithiocarbonate, 2-cyano-2-propyl 4-cyanobenzodithioate, and the like.

In some embodiments, the block copolymer can be associated with a DNA molecule, in particular a nucleic acid construct of the present disclosure, via several methods including, electrostatic interaction, high affinity, non-covalent bond, avidin-streptavidin conjugation, or by direct covalent attachment through, for example, an amide bond. In some embodiments, the RAFT copolymer can be associated with a DNA molecule, in particular a nucleic acid construct of the present disclosure, via electrostatic interaction complexed with a biological molecule. In some embodiments, the block copolymer can be associated with a DNA molecule, in particular a nucleic acid construct of the present disclosure, via electrostatic interaction complexed with a biological molecule. In some embodiments, the block copolymer can be associated with a DNA molecule, in particular a nucleic acid construct of the present disclosure, via a high affinity, non-covalent bond, avidin-streptavidin conjugation. In some embodiments, the block copolymer can be associated with a DNA molecule, in particular a nucleic acid construct of the present disclosure, by direct covalent attachment through, for example, an amide bond.

The polymer nanoparticles described herein can be associated with a nucleic acid construct of the present disclosure via electrostatic interaction, avidin-streptavidin conjugation, or by direct covalent attachment. Exemplary interactions include: polymer nanoparticle (PNP) with positively charged corona in the case of electrostatic loading; nucleic acid constructs with negative charges due to the phosphate groups; electrostatically loaded PNP-nucleic acid construct complexes; carboxylate group on the terminal end of the polymer chains in the corona of the PNP; primary amine group on the 5′ end of the amine terminated nucleic acid construct; phosphate group on the 3′ end of the nucleic acid construct; amide bond formed in the direct amidification reaction between the amine terminal nucleic acid construct and the carboxylate terminated PNP; primary amine on the biotin bonding protein such as avidin; amide bond formed between the carboxylate group on the terminal end of the polymer chains in the corona of the PNP and the primary amine on the biotin bonding protein such as avidin; nucleic acid construct with a biotin functional group on the 5′ terminus; electrostatic coupling reaction that occurs when positively charged PNPs are mixed with negatively charged nucleic acid constructs; direct amidification reaction that is carried out via an EDA-NHC reaction between the carboxylate group on the terminal end of the polymer chains in the corona of the PNP and the primary amine on the amine terminated nucleic acid constructs; direct amidification reaction that is carried out via an EDA-NHC reaction between the carboxylate group on the terminal end of the polymer chains in the corona of the PNP and the primary amine on the biotin bonding protein such as avidin; coupling of the biotin on the 5′ end of the nucleic acid construct and the avidin conjugated to the carboxylate terminus on the corona of the PNPs.

In another illustrative embodiment, the polymer nanoparticle composition can be coated with one or more polymers to protect the compositions from immune responses or to enhance endosomal escape. In one embodiment, the one or more polymers used for coating comprise polyethylene glycol. In another embodiment, the one or more polymers used for coating comprise polyethylene glycol poly-L-lysine. In yet another embodiment, the one or more polymers used for coating comprise polyethylenimine. In an additional embodiment, the one or more polymers used for coating comprise polyethylene glycol poly-L-lysine and polyethylenimine.

It will be appreciated that tuning the parameters and properties of the block copolymers described herein can be advantageous to their use in the compositions and methods as described herein. Accordingly, the methods for preparing block copolymers either in singleton or in library format as described herein are capable of providing particular parameters and properties of the block copolymers.

In some embodiments, a single chain transfer agent can be used in the RAFT polymerization process in connection with the present disclosure. In some embodiments, for a block polymer having more than one block, one or more single chain transfer agents can be used in the RAFT polymerization process in connection with the present disclosure. In some embodiments, for a block polymer having two blocks, a first chain transfer agent and a second chain transfer agent (which can be the same or different) can be used at each step of the RAFT polymerization process in connection with the present disclosure. In some embodiments, for a block polymer having three blocks, a first chain transfer agent, a second chain transfer agent, and a third chain transfer agent (which can be the same or different) can be used at each step of the RAFT polymerization process in connection with the present disclosure.

It will be appreciated that a variety of solvents can be used in the RAFT polymerization method steps and purification steps described herein. Suitable solvents include, but are not limited to, 2-Chloroethanol, Acetic Acid (Glacial), Acetone, Acetonitrile, Acetophenone, Aniline, Benzaldehyde, Benzyl Acetate, Carbon disulfide, Cyclohexane, Cyclohexanol, Di(ethylene glycol), Di(propylene glycol), Diacetone alcohol, Diethyl ether, Dimethylsulfoxide, Ethanol, Ethyl acetate, Ethylene glycol, Formaldehyde (37% solution), Formamide, Formic acid, Formic acid (96%), HexaneIsobutanol, Isopropanol, Isopropyl acetate, Isopropyl ether, m-Cresol, Methanol, Methyl acetate, Methyl ethyl ketone, Mineral Oil, N,N-Dimethylformamide, n-Butanol, n-Octane, n-Propanol, Propylene glycol, Pyridine, t-Butanol, Tetrahydrofuran, Trifluoroacetic acid, water, and the like, and combinations thereof.

While certain illustrative embodiments have been described in detail in the drawings and the foregoing description, such an illustration and description is to be considered as exemplary and not restrictive in character, it being understood that only illustrative embodiments have been shown and described and that all changes and modifications that come within the spirit of the disclosure are desired to be protected. There exist a plurality of advantages of the present disclosure arising from the various features of the apparatus, systems, and methods described herein. It will be noted that alternative embodiments of the apparatus, systems, and methods of the present disclosure may not include all of the features described, yet still benefit from at least some of the advantages of such features. Those of ordinary skill in the art may readily devise their own implementations of the apparatus, systems, and methods that incorporate one or more of the features of the present disclosure.

### EXAMPLES

### Example 1

### Polymer Nanoparticle Synthesis

Diblock copolymers were synthesized similarly as described in U.S. Patent Application Publication No. 2022/0175812, the entirety of which is incorporated by reference herein, with some modifications using reversible addition-fragmentation chain transfer (RAFT) polymerization with reagents and amounts listed in Table X. Block 1 reagents were combined in either 50 mL conical tubes or 50 mL round-bottom flasks, purged with argon, and heated to 70° C. for 15-16 hours or 24 hours using either an oil bath or a VWR 1400E vacuum oven. The reaction products were purified using either dialysis for at least 2 days or four 80:20 pentane:ether precipitation washes and centrifugation cycles and dried in vacuo. The Block 1 products were used as the macroRAFT agents for Block 2, and the calculated reagent volumes (as calculated based on theoretical molecular weight information for Block 1) were combined in Corning 96-well polypropylene cluster tubes for the Block 2 reactions. The reaction mixtures were argon purged before being heated at 60-70° C. for 24 hours. The reaction products were purified using either four 80:20 pentane:ether precipitation washes and centrifugation cycles or three 80:20 isopropanol:water washes and centrifugation cycles followed by drying in vacuo. Finally, the purified materials were lyophilized for 3-4 days and stored at room temperature for experimental use.

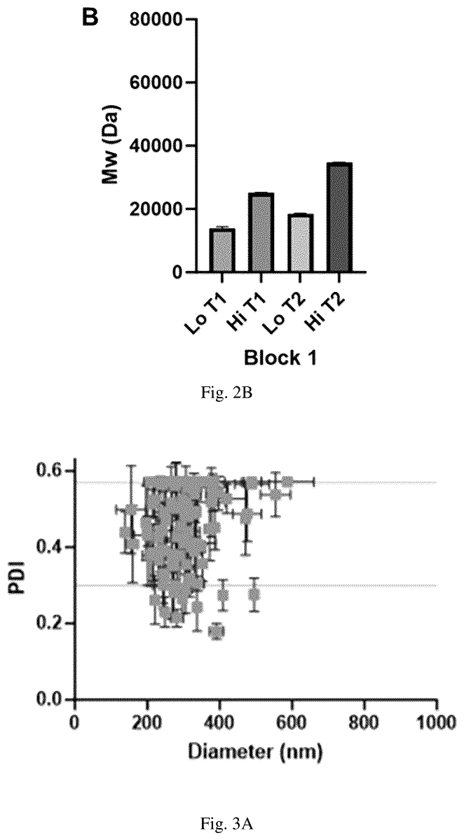

FIGS. 2A and 2B depict Block 1 polymer characterization. FIG. 2A shows a bar graph of the percent monomer conversion as measured by proton nuclear magnetic resonance (NMR). FIG. 2B shows a bar graph of molecular weight as measured by a conventional polystyrene standard curve gel permeation chromatography (GPC) method. Reagents are shown in Tables 1-5.

TABLE 1
Reagents and amounts used to synthesize macro-chain transfer
agents containing 2-dimethylaminoethyl methacrylate (DMAEMA)

| | 2-dimethyl | | (4-cyano-4-(((ethylthio) | | | | | | |
|---|---|---|---|---|---|---|---|---|---|
| | aminoethyl | | carbonothioyl)thio) | | Azobis- | | Dimethyl- | | Block 1 |
| | methacrylate | | pentanoicacid) | | isobutyronitrile | | formamide | | Reaction |
| Reagent | (DMAEMA) | | (ECT) | | (AIBN) | | (DMF) | | Yield |
| Purpose | Monomer | | Chain transfer agent | | Initiator | | Solvent | | % Yield |
| Batch 1 | 2985.6 | mg | 12.5 | mg | 7.8 | mg | 3579.7 | mg | 87.8% |
| Batch 2 | 2985.6 | mg | 10.5 | mg | 6.6 | mg | 3655.2 | mg | 90.0% |
| Batch 3 | 2994.9 | mg | 9.1 | mg | 5.7 | mg | 3702.4 | mg | 85.9% |
| Batch 4 | 2994.9 | mg | 8.0 | mg | 5.0 | mg | 3744.9 | mg | 92.2% |
| Batch 5 | 2994.9 | mg | 7.1 | mg | 4.5 | mg | 3778.0 | mg | 90.8% |
| Batch 6 | 2994.9 | mg | 6.4 | mg | 4.0 | mg | 3801.6 | mg | 90.9% |
| Batch 7 | 2994.9 | mg | 5.9 | mg | 3.7 | mg | 3825.2 | mg | 89.0% |
| Batch 8 | 2994.9 | mg | 5.4 | mg | 3.4 | mg | 3844.1 | mg | 90.3% |
| Batch 9 | 2994.9 | mg | 5.0 | mg | 3.1 | mg | 3858.3 | mg | 88.4% |
| Batch 10 | 2994.9 | mg | 4.7 | mg | 2.9 | mg | 3872.5 | mg | 87.7% |
| Batch 11 | 2994.9 | mg | 4.4 | mg | 2.7 | mg | 3881.9 | mg | 88.0% |
| Batch 12 | 2994.9 | mg | 4.1 | mg | 2.6 | mg | 3896.1 | mg | 87.2% |
| Batch 13 | 2985.6 | mg | 12.5 | mg | 7.8 | mg | 3579.7 | mg | 90.1% |
| Batch 14 | 2994.9 | mg | 8.0 | mg | 5.0 | mg | 3743.1 | mg | 91.9% |
| Batch 15 | 2994.9 | mg | 5.4 | mg | 3.4 | mg | 3843.2 | mg | 88.8% |
| Batch 16 | 2994.9 | mg | 4.1 | mg | 2.6 | mg | 3894.2 | mg | 87.0% |
| Batch 17 | 2985.6 | mg | 12.5 | mg | 7.8 | mg | 2229.0 | mg | 96.9% |
| Batch 18 | 2985.6 | mg | 10.5 | mg | 6.6 | mg | 2521.8 | mg | 95.6% |
| Batch 19 | 2994.9 | mg | 8.0 | mg | 5.0 | mg | 2880.7 | mg | 95.9% |
| Batch 20 | 2994.9 | mg | 5.4 | mg | 3.4 | mg | 3258.5 | mg | 87.8% |
| Batch 21 | 14834.7 | mg | 142.4 | mg | 88.6 | mg | 26285.4 | mg | 76.8% |
| Batch 22 | 14928 | mg | 71.6 | mg | 44.6 | mg | 26191.0 | mg | 68.6% |
| Batch 23 | 14834.7 | mg | 142.4 | mg | 88.6 | mg | 26285.4 | mg | 94.5% |
| Batch 24 | 14928 | mg | 71.6 | mg | 44.6 | mg | 26191.0 | mg | 95.6% |

TABLE 2
Reagents and amounts used to synthesize macro-chain transfer agents
containing 2-amino ethyl methacrylate hydrochloride (AEMA)

| | | | (4-cyano-4-(((ethylthio) | | | | | | |
|---|---|---|---|---|---|---|---|---|---|
| | 2-amino ethyl | | carbonothioyl)thio) | | 4,4′-Azobis(4- | | | | Block 1 |
| | methacrylate | | pentanoicacid) | | cyanovaleric acid) | | 1M pH 5.2 | | Reaction |
| Reagent | hydrochloride (AEMA) | | (ECT) | | (ACVA) | | acetate buffer | | Yield |
| Purpose | Monomer | | Chain transfer agent | | Initiator | | Solvent | | % Yield |
| Batch 1 | 2909.9 | mg | 93.0 | mg | 19.5 | mg | 17.7 | mL | 20.6 |
| Batch 2 | 2966.8 | mg | 37.6 | mg | 8.3 | mg | 18.0 | mL | 20.1 |
| Batch 3 | 2974.2 | mg | 23.7 | mg | 5.1 | mg | 18.1 | mL | 20.1 |
| Batch 4 | 2979.4 | mg | 17.4 | mg | 4.0 | mg | 18.1 | mL | 20.1 |
| Batch 5 | 2986.4 | mg | 13.9 | mg | 2.9 | mg | 18.1 | mL | 20.4 |
| Batch 6 | 2990.5 | mg | 11.7 | mg | 2.4 | mg | 18.2 | mL | 0.209 |
| Batch 7 | 2995.2 | mg | 9.9 | mg | 2.0 | mg | 18.2 | mL | 0.207 |

TABLE 3
Reagents and amounts used to synthesize the second block

| | | | | | | | | | Block 1 macroRAFT agent, | | | | |
|---|---|---|---|---|---|---|---|---|---|---|---|---|---|
| | | | | | | | | | meaning ECT + DMAEMA or | | | | |
| | | | | | methyl | ethyl | poly(ethylene | | ECT + AEMA | | | | |
| | 2-dimethylamino | methyl | | butyl | acrylic | acrylic | glycol) | | | | Macro | | Block 2 |
| | ethyl acrylate | methacrylate | | methacrylate | acid | acid | methacrylate | | Macro Chain | | Chain | | Reaction |
| PNP | (DMAEMA) | (MMA) | | (BMA) | (MAA) | (EAA) | (PEGMA) | | transfer | | transfer | | Mass Yield |
| ID | Monomer | Monomer | | Monomer | Monomer | Monomer | Monomer | | agent MW | | agent mass | | % Yield |
| 1 | N/A | 46 | μL | N/A | N/A | N/A | N/A | | 18513 | Da | 16.8 | mg | 21.6% |
| 2 | N/A | N/A | | 43 μL | N/A | N/A | N/A | | 40985 | Da | 21.9 | mg | 79.6% |
| 3 | N/A | N/A | | 39 μL | N/A | N/A | N/A | | 40985 | Da | 25.2 | mg | 60.6% |
| 4 | N/A | 28 | μL | N/A | N/A | N/A | N/A | | 34701 | Da | 33.0 | mg | 38.6% |
| 5 | N/A | 39 | μL | N/A | N/A | N/A | N/A | | 34701 | Da | 23.4 | mg | 28.1% |
| 6 | N/A | 46 | μL | N/A | N/A | N/A | N/A | | 18513 | Da | 16.8 | mg | 21.6% |
| 7 | N/A | 39 | μL | N/A | N/A | N/A | N/A | | 13961 | Da | 23.4 | mg | 15.2% |
| 8 | N/A | 38 | μL | N/A | N/A | N/A | N/A | | 34701 | Da | 24.3 | mg | 48.0% |
| 9 | N/A | 40 | μL | N/A | N/A | N/A | N/A | | 34701 | Da | 22.8 | mg | 54.2% |
| 10 | N/A | 32 | μL | N/A | N/A | N/A | N/A | | 40985 | Da | 30.0 | mg | 59.3% |
| 11 | N/A | N/A | | 38 μL | N/A | N/A | N/A | | 40985 | Da | 30.0 | mg | 56.0% |
| 12 | N/A | 33 | μL | N/A | N/A | N/A | N/A | | 40985 | Da | 29.1 | mg | 50.2% |
| 13 | N/A | N/A | | 42 μL | N/A | N/A | N/A | | 40985 | Da | 22.5 | mg | 57.4% |
| 14 | N/A | 46 | μL | N/A | N/A | N/A | N/A | | 13961 | Da | 13.5 | mg | 18.5% |
| 15 | N/A | 50 | μL | N/A | N/A | N/A | N/A | | 13961 | Da | 13.5 | mg | 45.4% |
| 16 | N/A | 54 | μL | N/A | N/A | N/A | N/A | | 25084 | Da | 20.7 | mg | 37.6% |
| 17 | N/A | 42 | μL | N/A | N/A | N/A | N/A | | 25084 | Da | 20.7 | mg | 35.9% |
| 18 | N/A | 60 | μL | N/A | N/A | N/A | N/A | | 34701 | Da | 25.2 | mg | 33.3% |
| 19 | N/A | 37 | μL | N/A | N/A | N/A | N/A | | 34701 | Da | 25.2 | mg | 39.2% |
| 20 | N/A | 58 | μL | N/A | N/A | N/A | N/A | | 18513 | Da | 15.9 | mg | 30.9% |
| 21 | N/A | 65 | μL | N/A | N/A | N/A | N/A | | 18513 | Da | 15.3 | mg | 32.5% |
| 22 | N/A | 72 | μL | N/A | N/A | N/A | N/A | | 18513 | Da | 14.7 | mg | 11.0% |
| 23 | N/A | 47 | μL | N/A | N/A | N/A | N/A | | 18513 | Da | 15.9 | mg | 25.0% |
| 24 | N/A | 48 | μL | N/A | N/A | N/A | N/A | | 18513 | Da | 15.3 | mg | 21.2% |
| 25 | N/A | 49 | μL | N/A | N/A | N/A | N/A | | 18513 | Da | 14.7 | mg | 16.5% |
| 26 | N/A | 40 | μL | N/A | N/A | N/A | N/A | | 34701 | Da | 22.8 | mg | 0.2% |
| 27 | N/A | 39 | μL | N/A | N/A | N/A | N/A | | 13961 | Da | 23.4 | mg | 66.8% |
| 28 | N/A | 31 | μL | N/A | N/A | N/A | N/A | | 25084 | Da | 30.0 | mg | 38.9% |
| 29 | N/A | 20 | μL | N/A | N/A | N/A | N/A | | 25084 | Da | 30.0 | mg | 44.6% |
| 30 | N/A | 37 | μL | N/A | N/A | N/A | N/A | | 18513 | Da | 25.5 | mg | 62.0% |
| 31 | N/A | 24 | μL | N/A | N/A | N/A | N/A | | 18513 | Da | 25.5 | mg | 40.7% |
| 32 | N/A | 18 | μL | N/A | N/A | N/A | N/A | | 34701 | Da | 33.0 | mg | 40.2% |
| 33 | N/A | 24 | μL | N/A | N/A | N/A | N/A | | 13961 | Da | 22.5 | mg | 42.6% |
| 34 | N/A | 23 | μL | N/A | N/A | N/A | N/A | | 13961 | Da | 21.6 | mg | 23.3% |
| 35 | N/A | 22 | μL | N/A | N/A | N/A | N/A | | 13961 | Da | 21.0 | mg | 31.5% |
| 36 | N/A | 29 | μL | N/A | N/A | N/A | N/A | | 34701 | Da | 33.0 | mg | 52.8% |
| 37 | N/A | 30 | μL | N/A | N/A | N/A | N/A | | 34701 | Da | 33.0 | mg | 56.8% |
| 38 | N/A | 31 | μL | N/A | N/A | N/A | N/A | | 34701 | Da | 30.0 | mg | 43.4% |
| 39 | N/A | 42 | μL | N/A | N/A | N/A | N/A | | 21127 | Da | 20.7 | mg | 47.9% |
| 40 | N/A | N/A | | 49 μL | N/A | N/A | N/A | | 21127 | Da | 16.2 | mg | 63.0% |
| 41 | N/A | 45 | μL | N/A | N/A | N/A | N/A | | 21127 | Da | 18.3 | mg | 56.9% |
| 42 | N/A | N/A | | 51 μL | N/A | N/A | N/A | | 21127 | Da | 14.4 | mg | 54.1% |
| 43 | N/A | 31 | μL | N/A | N/A | N/A | N/A | | 40985 | Da | 30.0 | mg | 60.5% |
| 44 | N/A | 45 | μL | N/A | N/A | N/A | N/A | | 21127 | Da | 17.7 | mg | 16.8% |
| 45 | N/A | N/A | | 52 μL | N/A | N/A | N/A | | 21127 | Da | 13.8 | mg | 70.7% |
| 46 | N/A | 47 | μL | N/A | N/A | N/A | N/A | | 21127 | Da | 15.6 | mg | 79.9% |
| 47 | N/A | N/A | | 54 μL | N/A | N/A | N/A | | 21127 | Da | 12.0 | mg | 70.5% |
| 48 | N/A | 35 | μL | N/A | N/A | N/A | N/A | | 40985 | Da | 27.0 | mg | 67.2% |
| 49 | N/A | 35 | μL | N/A | N/A | N/A | N/A | | 40985 | Da | 27.6 | mg | 80.9% |
| 50 | N/A | N/A | | 41 μL | N/A | N/A | N/A | | 40985 | Da | 23.4 | mg | 63.2% |
| 51 | N/A | N/A | | 44 μL | N/A | N/A | N/A | | 40985 | Da | 20.7 | mg | 58.4% |
| 52 | N/A | 22 | μL | N/A | N/A | N/A | N/A | | 29995 | Da | 19.2 | mg | N/A |
| 53 | N/A | 24 | μL | N/A | N/A | N/A | N/A | | 29995 | Da | 17.4 | mg | N/A |
| 54 | N/A | 26 | μL | N/A | N/A | N/A | N/A | | 29995 | Da | 15.8 | mg | N/A |
| 55 | N/A | 27 | μL | N/A | N/A | N/A | N/A | | 29995 | Da | 14.4 | mg | N/A |
| 56 | N/A | 28 | μL | N/A | N/A | N/A | N/A | | 29995 | Da | 13.4 | mg | N/A |
| 57 | N/A | 30 | μL | N/A | N/A | N/A | N/A | | 29995 | Da | 12.4 | mg | N/A |
| 58 | N/A | 31 | μL | N/A | N/A | N/A | N/A | | 29995 | Da | 10.8 | mg | 25.2% |
| 59 | N/A | 30 | μL | N/A | N/A | N/A | N/A | | 29995 | Da | 12.4 | mg | N/A |
| 60 | N/A | 21 | μL | N/A | N/A | N/A | N/A | | 42125 | Da | 20.0 | mg | N/A |
| 61 | N/A | 28 | μL | N/A | N/A | N/A | N/A | | 42125 | Da | 13.8 | mg | N/A |
| 62 | N/A | 19 | μL | N/A | N/A | N/A | N/A | | 42125 | Da | 22.0 | mg | N/A |
| 63 | N/A | 23 | μL | N/A | N/A | N/A | N/A | | 50629 | Da | 16.6 | mg | N/A |
| 64 | 4.8 μL | 14 | μL | N/A | 2.4 μL | N/A | N/A | | 42125 | Da | 19.8 | mg | 75.1% |
| 65 | 7.7 μL | 14 | μL | N/A | 0.8 μL | N/A | N/A | | 42125 | Da | 19.2 | mg | 78.3% |
| 66 | 2.5 μL | 5.9 | μL | N/A | 2.6 μL | N/A | N/A | | 58681 | Da | 30.0 | mg | 79.7% |
| 67 | N/A | 4.1 | μL | N/A | N/A | N/A | N/A | | 47481 | Da | 36.0 | mg | N/A |
| 68 | N/A | 21 | μL | N/A | N/A | N/A | N/A | | 47481 | Da | 20.0 | mg | 83.3% |
| 69 | N/A | 22 | μL | N/A | N/A | N/A | N/A | | 9919 | Da | 20.0 | mg | 48.7% |
| 70 | N/A | 36 | μL | N/A | N/A | N/A | N/A | | 9919 | Da | 6.6 | mg | 36.6% |
| 71 | N/A | 28 | μL | N/A | N/A | N/A | N/A | | 26262 | Da | 13.8 | mg | 21.1% |
| 72 | N/A | 25 | μL | N/A | N/A | N/A | N/A | | 34422 | Da | 16.2 | mg | 40.1% |
| 73 | N/A | 13 | μL | N/A | N/A | N/A | N/A | | 34422 | Da | 28.0 | mg | N/A |
| 74 | N/A | 9 | μL | N/A | N/A | N/A | N/A | | 56390 | Da | 32.0 | mg | N/A |
| 75 | N/A | 36 | μL | N/A | N/A | N/A | N/A | | 9919 | Da | 6.2 | mg | 32.2% |
| 76 | N/A | 26 | μL | N/A | N/A | N/A | N/A | | 34422 | Da | 15.4 | mg | 51.1% |
| 77 | N/A | 11 | μL | N/A | N/A | N/A | N/A | | 56390 | Da | 30.0 | mg | N/A |
| 78 | N/A | 27 | μL | N/A | N/A | N/A | N/A | | 34422 | Da | 14.6 | mg | 52.2% |
| 79 | N/A | 31 | μL | N/A | N/A | N/A | N/A | | 9919 | Da | 11.4 | mg | 60.0% |
| 80 | N/A | 21 | μL | N/A | N/A | N/A | N/A | | 26262 | Da | 20.0 | mg | 66.1% |
| 81 | N/A | 18 | μL | N/A | N/A | N/A | N/A | | 34422 | Da | 24.0 | mg | 84.7% |
| 82 | N/A | 13 | μL | N/A | N/A | N/A | N/A | | 56390 | Da | 28.0 | mg | N/A |
| 83 | N/A | 31 | μL | N/A | N/A | N/A | N/A | | 26262 | Da | 11.6 | mg | 32.7% |
| 84 | N/A | 25 | μL | N/A | N/A | N/A | N/A | | 47481 | Da | 16.8 | mg | 53.2% |
| 85 | N/A | 26 | μL | N/A | N/A | N/A | N/A | | 47481 | Da | 16.2 | mg | 51.0% |
| 86 | N/A | 24 | μL | N/A | N/A | N/A | N/A | | 26262 | Da | 17.2 | mg | 56.6% |
| 87 | N/A | 32 | μL | N/A | N/A | N/A | N/A | | 26262 | Da | 10.4 | mg | 15.6% |
| 88 | N/A | 24 | μL | N/A | N/A | N/A | N/A | | 56390 | Da | 17.2 | mg | 43.7% |
| 89 | 8.8 μL | 14 | μL | N/A | N/A | 8.3 μL | N/A | | 32669 | Da | 10.8 | mg | 47.8% |
| 90 | 2.3 μL | 10 | μL | N/A | N/A | 2.7 μL | N/A | | 37962 | Da | 26.0 | mg | 73.1% |
| 91 | 4.5 μL | 7.1 | μL | N/A | N/A | 4.0 μL | N/A | | 37962 | Da | 26.0 | mg | 70.8% |
| 92 | 4.5 μL | 8.5 | μL | N/A | N/A | 2.7 μL | N/A | | 37962 | Da | 26.0 | mg | 70.3% |
| 93 | 0.6 μL | 3.1 | μL | N/A | N/A | 0.4 μL | N/A | | 50311 | Da | 36.0 | mg | 77.6% |
| 94 | N/A | 16 | μL | N/A | 5.3 μL | N/A | N/A | | 37962 | Da | 19.8 | mg | 79.3% |
| 95 | N/A | 8.2 | μL | N/A | N/A | N/A | 11.0 | μL | 37962 | Da | 20.0 | mg | 86.7% |
| 96 | N/A | 7.6 | μL | N/A | N/A | N/A | 9.9 | μL | 43305 | Da | 22.0 | mg | 80.4% |
| 97 | N/A | 7.0 | μL | N/A | N/A | N/A | 9.1 | μL | 50311 | Da | 24.0 | mg | 87.1% |
| 98 | N/A | 12 | μL | N/A | N/A | N/A | 9.4 | μL | 37962 | Da | 18.2 | mg | N/A |
| 99 | N/A | 9.3 | μL | N/A | N/A | N/A | 12.0 | μL | 43305 | Da | 18.0 | mg | 73.2% |

Block 1 is a DMAEMA homopolymer for PNPs 1-66 and 89-99. Block 1 is an AEMA homopolymer for PNPs 67-88.

TABLE 4
Initiator and solvent amount, and yield for PNPs 52-99

| | Block 1 macroRAFT | | | | | | | |
|---|---|---|---|---|---|---|---|---|
| | agent, meaning | | | | | | | |
| | ECT + DMAEMA | | | | | | | |
| | or ECT + AEMA | | Azobisiso- | 4,4′-Azobis(4- | Dimethyl- | | | Block 2 |
| | Macro Chain | Macro Chain | butyronitrile | cyanovaleric | formamide | | | Reaction |
| PNP | transfer agent | transfer agent | (AIBN) | acid) (ACVA) | (DMF) | | Water | Mass Yield |
| ID | MW | mass | Initiator | Initiator | Solvent | | Solvent | % Yield |
| 52 | 29995 Da | 19.2 mg | 0.105 mg | N/A | 78 | μL | N/A | N/A |
| 53 | 29995 Da | 17.4 mg | 0.096 mg | N/A | 86 | μL | N/A | N/A |
| 54 | 29995 Da | 15.8 mg | 0.087 mg | N/A | 92 | μL | N/A | N/A |
| 55 | 29995 Da | 14.4 mg | 0.078 mg | N/A | 98 | μL | N/A | N/A |
| 56 | 29995 Da | 13.4 mg | 0.072 mg | N/A | 100 | μL | N/A | N/A |
| 57 | 29995 Da | 12.4 mg | 0.069 mg | N/A | 110 | μL | N/A | N/A |
| 58 | 29995 Da | 10.8 mg | 0.060 mg | N/A | 110 | μL | N/A | 25.2% |
| 59 | 29995 Da | 12.4 mg | 0.014 mg | N/A | 110 | μL | N/A | N/A |
| 60 | 42125 Da | 20.0 mg | 0.081 mg | N/A | 73 | μL | N/A | N/A |
| 61 | 42125 Da | 13.8 mg | 0.054 mg | N/A | 100 | μL | N/A | N/A |
| 62 | 42125 Da | 22.0 mg | 0.018 mg | N/A | 68 | μL | N/A | N/A |
| 63 | 50629 Da | 16.6 mg | 0.060 mg | N/A | 83 | μL | N/A | N/A |
| 64 | 42125 Da | 19.8 mg | 0.078 mg | N/A | 77 | μL | N/A | 75.1% |
| 65 | 42125 Da | 19.2 mg | 0.075 mg | N/A | 79 | μL | N/A | 78.3% |
| 66 | 58681 Da | 30.0 mg | 0.084 mg | N/A | 39 | μL | N/A | 79.7% |

| | | | | | | | | |
|---|---|---|---|---|---|---|---|---|
| 67 | 47481 Da | 36.0 mg | N/A | 0.213 mg | N/A | 7.9 | μL | N/A |
| 68 | 47481 Da | 20.0 mg | N/A | 0.120 mg | N/A | 72 | μL | 83.3% |
| 69 | 9919 Da | 20.0 mg | N/A | 0.571 mg | N/A | 60 | μL | 48.7% |
| 70 | 9919 Da | 6.6 mg | N/A | 0.186 mg | N/A | 120 | μL | 36.6% |
| 71 | 26262 Da | 13.8 mg | N/A | 0.147 mg | N/A | 98 | μL | 21.1% |
| 72 | 34422 Da | 16.2 mg | N/A | 0.132 mg | N/A | 89 | μL | 40.1% |
| 73 | 34422 Da | 28.0 mg | N/A | 0.228 mg | N/A | 40 | μL | N/A |
| 74 | 56390 Da | 32.0 mg | N/A | 0.156 mg | N/A | 28 | μL | N/A |
| 75 | 9919 Da | 6.2 mg | N/A | 0.174 mg | N/A | 130 | μL | 32.2% |
| 76 | 34422 Da | 15.4 mg | N/A | 0.126 mg | N/A | 93 | μL | 51.1% |
| 77 | 56390 Da | 30.0 mg | N/A | 0.147 mg | N/A | 36 | μL | N/A |
| 78 | 34422 Da | 14.6 mg | N/A | 0.120 mg | N/A | 96 | μL | 52.2% |
| 79 | 9919 Da | 11.4 mg | N/A | 0.331 mg | N/A | 100 | μL | 60.0% |
| 80 | 26262 Da | 20.0 mg | N/A | 0.219 mg | N/A | 69 | μL | 66.1% |
| 81 | 34422 Da | 24.0 mg | N/A | 0.189 mg | N/A | 60 | μL | 84.7% |
| 82 | 56390 Da | 28.0 mg | N/A | 0.138 mg | N/A | 44 | μL | N/A |
| 83 | 26262 Da | 11.6 mg | N/A | 0.123 mg | N/A | 110 | μL | 32.7% |
| 84 | 47481 Da | 16.8 mg | N/A | 0.099 mg | N/A | 88 | μL | 53.2% |
| 85 | 47481 Da | 16.2 mg | N/A | 0.096 mg | N/A | 91 | μL | 51.0% |
| 86 | 26262 Da | 17.2 mg | N/A | 0.183 mg | N/A | 84 | μL | 56.6% |
| 87 | 26262 Da | 10.4 mg | N/A | 0.111 mg | N/A | 110 | μL | 15.6% |
| 88 | 56390 Da | 17.2 mg | N/A | 0.084 mg | N/A | 87 | μL | 43.7% |

| | | | | | | | | |
|---|---|---|---|---|---|---|---|---|
| 89 | 32669 Da | 10.8 mg | 0.056 mg | N/A | 110 | μL | N/A | 47.8% |
| 90 | 37962 Da | 26.0 mg | 0.112 mg | N/A | 47 | μL | N/A | 73.1% |
| 91 | 37962 Da | 26.0 mg | 0.109 mg | N/A | 49 | μL | N/A | 70.8% |
| 92 | 37962 Da | 26.0 mg | 0.109 mg | N/A | 49 | μL | N/A | 70.3% |
| 93 | 50311 Da | 36.0 mg | 0.118 mg | N/A | 5.1 | μL | N/A | 77.6% |
| 94 | 37962 Da | 19.8 mg | 0.086 mg | N/A | 72 | μL | N/A | 79.3% |
| 95 | 37962 Da | 20.0 mg | 0.089 mg | N/A | 71 | μL | N/A | 86.7% |
| 96 | 43305 Da | 22.0 mg | 0.083 mg | N/A | 66 | μL | N/A | 80.4% |
| 97 | 50311 Da | 24.0 mg | 0.077 mg | N/A | 60 | μL | N/A | 87.1% |
| 98 | 37962 Da | 18.2 mg | 0.078 mg | N/A | 81 | μL | N/A | N/A |
| 99 | 43305 Da | 18.0 mg | 0.068 mg | N/A | 83 | μL | N/A | 73.2% |

**Block 1 macroRAFT agent, meaning ECT + DMAEMA or ECT + AEMA. The ECT end groups (R & Z) were still present to perform their function, but they were on the end of the p(DMAEMA) or p(AEMA) polymer synthesized as Block 1. For reference, here is ECT showing the R & Z groups on either side of the trithiocarbonyl group.

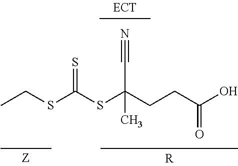

TABLE 5
Block 1 amount and C:I ratio for PNPs 1-99

| PNP | Block 1 | |
|---|---|---|
| ID | mCTA Mw | C:I |
| ID | (Da) | mCTA:I |
| 1 | 18513 | 5 |
| 2 | 40985 | 1 |
| 3 | 40985 | 1 |
| 4 | 34701 | 1 |
| 5 | 34701 | 5 |
| 6 | 18513 | 1 |
| 7 | 30619 | 5 |
| 8 | 34701 | 5 |
| 9 | 34701 | 1 |
| 10 | 40985 | 1 |
| 11 | 40985 | 1 |
| 12 | 40985 | 1 |
| 13 | 40985 | 1 |
| 14 | 13961 | 1 |
| 15 | 13961 | 5 |
| 16 | 25084 | 1 |
| 17 | 25084 | 5 |
| 18 | 34701 | 1 |
| 19 | 34701 | 5 |
| 20 | 18513 | 1 |
| 21 | 18513 | 1 |
| 22 | 18513 | 1 |
| 23 | 18513 | 5 |
| 24 | 18513 | 5 |
| 25 | 18513 | 5 |
| 26 | 34701 | 5 |
| 27 | 13961 | 1 |
| 28 | 25084 | 5 |
| 29 | 25084 | 5 |
| 30 | 18513 | 1 |
| 31 | 18513 | 5 |
| 32 | 34701 | 5 |
| 33 | 13961 | 5 |
| 34 | 13961 | 5 |
| 35 | 13961 | 5 |
| 36 | 34701 | 1 |
| 37 | 34701 | 1 |
| 38 | 34701 | 1 |
| 39 | 21127 | 1 |
| 40 | 21127 | 1 |
| 41 | 21127 | 1 |
| 42 | 21127 | 1 |
| 43 | 40985 | 1 |
| 44 | 21127 | 1 |
| 45 | 21127 | 1 |
| 46 | 21127 | 1 |
| 47 | 21127 | 1 |
| 48 | 40985 | 1 |
| 49 | 40985 | 1 |
| 50 | 40985 | 1 |
| 51 | 40985 | 1 |
| 52 | 29995 | 1 |
| 53 | 29995 | 1 |
| 54 | 29995 | 1 |
| 55 | 29995 | 1 |
| 56 | 29995 | 1 |
| 57 | 29995 | 1 |
| 58 | 29995 | 1 |
| 59 | 29995 | 5 |
| 60 | 42125 | 1 |
| 61 | 42125 | 1 |
| 62 | 42125 | 5 |
| 63 | 50629 | 1 |
| 64 | 42125 | 1 |
| 65 | 42125 | 1 |
| 66 | 58681 | 1 |
| 67 | 47481 | 1 |
| 68 | 47481 | 1 |
| 69 | 9919 | 1 |
| 70 | 9919 | 1 |
| 71 | 26262 | 1 |
| 72 | 34422 | 1 |
| 73 | 34422 | 1 |
| 74 | 56390 | 1 |
| 75 | 9919 | 1 |
| 76 | 34422 | 1 |
| 77 | 56390 | 1 |
| 78 | 34422 | 1 |
| 79 | 9919 | 1 |
| 80 | 26262 | 1 |
| 81 | 34422 | 1 |
| 82 | 56390 | 1 |
| 83 | 26262 | 1 |
| 84 | 47481 | 1 |
| 85 | 47481 | 1 |
| 86 | 26262 | 1 |
| 87 | 26262 | 1 |
| 88 | 56390 | 1 |
| 89 | 32669 | 1 |
| 90 | 37962 | 1 |
| 91 | 37962 | 1 |
| 92 | 37962 | 1 |
| 93 | 50311 | 1 |
| 94 | 37962 | 1 |
| 95 | 37962 | 1 |
| 96 | 43305 | 1 |
| 97 | 50311 | 1 |
| 98 | 37962 | 1 |
| 99 | 43305 | 1 |

### Example 2

### PNP Summary

A summary of the polymers corresponding to PNPs 1-51 is provided in Table 6. The molecular weight was determined by a conventional polystyrene standard curve gel permeation chromatography (GPC) method using a Malvern OMNISEC Gel Permeation Chromatograph equipped with refractive index, UV/Vis, light scattering, and viscosity detectors to characterize the molecular weight of polymers. Commercially available narrow distribution polystyrene standards with known molecular weights were used to create a molecular weight standard curve, and this standard curve was used to determine the molecular weight of experimental polymer samples described herein.

TABLE 6
Composition of PNPs 1-99

| PNP | Block 1 | | | | | | | | |
|---|---|---|---|---|---|---|---|---|---|
| ID | mCTA | C:I | Block 2 Molar Composition | | | | | | |
| ID | Mw (Da) | mCTA:I | Monomer 1 | % Monomer 1 | Monomer 2 | % Monomer 2 | Monomer 3 | % Monomer 3 | Theoretical DP^{a} |
| 1 | 18513 | 5 | MMA | 100 | N/A | N/A | N/A | N/A | 480 |
| 2 | 40985 | 1 | BMA | 100 | N/A | N/A | N/A | N/A | 500 |
| 3 | 40985 | 1 | BMA | 100 | N/A | N/A | N/A | N/A | 400 |
| 4 | 34701 | 1 | MMA | 100 | N/A | N/A | N/A | N/A | 480 |
| 5 | 34701 | 5 | MMA | 100 | N/A | N/A | N/A | N/A | 540 |
| 6 | 18513 | 1 | MMA | 100 | N/A | N/A | N/A | N/A | 480 |
| 7 | 30619 | 5 | MMA | 100 | N/A | N/A | N/A | N/A | 480 |
| 8 | 34701 | 5 | MMA | 100 | N/A | N/A | N/A | N/A | 510 |
| 9 | 34701 | 1 | MMA | 100 | N/A | N/A | N/A | N/A | 570 |
| 10 | 40985 | 1 | MMA | 100 | N/A | N/A | N/A | N/A | 400 |
| 11 | 40985 | 1 | BMA | 100 | N/A | N/A | N/A | N/A | 400 |
| 12 | 40985 | 1 | MMA | 100 | N/A | N/A | N/A | N/A | 450 |
| 13 | 40985 | 1 | BMA | 100 | N/A | N/A | N/A | N/A | 500 |
| 14 | 13961 | 1 | MMA | 100 | N/A | N/A | N/A | N/A | 480 |
| 15 | 13961 | 5 | MMA | 100 | N/A | N/A | N/A | N/A | 480 |
| 16 | 25084 | 1 | MMA | 100 | N/A | N/A | N/A | N/A | 480 |
| 17 | 25084 | 5 | MMA | 100 | N/A | N/A | N/A | N/A | 480 |
| 18 | 34701 | 1 | MMA | 100 | N/A | N/A | N/A | N/A | 480 |
| 19 | 34701 | 5 | MMA | 100 | N/A | N/A | N/A | N/A | 480 |
| 20 | 18513 | 1 | MMA | 100 | N/A | N/A | N/A | N/A | 510 |
| 21 | 18513 | 1 | MMA | 100 | N/A | N/A | N/A | N/A | 540 |
| 22 | 18513 | 1 | MMA | 100 | N/A | N/A | N/A | N/A | 570 |
| 23 | 18513 | 5 | MMA | 100 | N/A | N/A | N/A | N/A | 510 |
| 24 | 18513 | 5 | MMA | 100 | N/A | N/A | N/A | N/A | 540 |
| 25 | 18513 | 5 | MMA | 100 | N/A | N/A | N/A | N/A | 570 |
| 26 | 34701 | 5 | MMA | 100 | N/A | N/A | N/A | N/A | 570 |
| 27 | 13961 | 1 | MMA | 100 | N/A | N/A | N/A | N/A | 480 |
| 28 | 25084 | 5 | MMA | 100 | N/A | N/A | N/A | N/A | 480 |
| 29 | 25084 | 5 | MMA | 100 | N/A | N/A | N/A | N/A | 480 |
| 30 | 18513 | 1 | MMA | 100 | N/A | N/A | N/A | N/A | 480 |
| 31 | 18513 | 5 | MMA | 100 | N/A | N/A | N/A | N/A | 480 |
| 32 | 34701 | 5 | MMA | 100 | N/A | N/A | N/A | N/A | 480 |
| 33 | 13961 | 5 | MMA | 100 | N/A | N/A | N/A | N/A | 510 |
| 34 | 13961 | 5 | MMA | 100 | N/A | N/A | N/A | N/A | 540 |
| 35 | 13961 | 5 | MMA | 100 | N/A | N/A | N/A | N/A | 570 |
| 36 | 34701 | 1 | MMA | 100 | N/A | N/A | N/A | N/A | 510 |
| 37 | 34701 | 1 | MMA | 100 | N/A | N/A | N/A | N/A | 540 |
| 38 | 34701 | 1 | MMA | 100 | N/A | N/A | N/A | N/A | 570 |
| 39 | 21127 | 1 | MMA | 100 | N/A | N/A | N/A | N/A | 400 |
| 40 | 21127 | 1 | BMA | 100 | N/A | N/A | N/A | N/A | 400 |
| 41 | 21127 | 1 | MMA | 100 | N/A | N/A | N/A | N/A | 400 |
| 42 | 21127 | 1 | BMA | 100 | N/A | N/A | N/A | N/A | 400 |
| 43 | 40985 | 1 | MMA | 100 | N/A | N/A | N/A | N/A | 400 |
| 44 | 21127 | 1 | MMA | 100 | N/A | N/A | N/A | N/A | 500 |
| 45 | 21127 | 1 | BMA | 100 | N/A | N/A | N/A | N/A | 500 |
| 46 | 21127 | 1 | MMA | 100 | N/A | N/A | N/A | N/A | 500 |
| 47 | 21127 | 1 | BMA | 100 | N/A | N/A | N/A | N/A | 500 |
| 48 | 40985 | 1 | MMA | 100 | N/A | N/A | N/A | N/A | 500 |
| 49 | 40985 | 1 | MMA | 100 | N/A | N/A | N/A | N/A | 500 |
| 50 | 40985 | 1 | BMA | 100 | N/A | N/A | N/A | N/A | 450 |
| 51 | 40985 | 1 | BMA | 100 | N/A | N/A | N/A | N/A | 550 |
| 52 | 29995 | 1 | MMA | 100 | N/A | N/A | N/A | N/A | 323 |
| 53 | 29995 | 1 | MMA | 100 | N/A | N/A | N/A | N/A | 391 |
| 54 | 29995 | 1 | MMA | 100 | N/A | N/A | N/A | N/A | 459 |
| 55 | 29995 | 1 | MMA | 100 | N/A | N/A | N/A | N/A | 527 |
| 56 | 29995 | 1 | MMA | 100 | N/A | N/A | N/A | N/A | 595 |
| 57 | 29995 | 1 | MMA | 100 | N/A | N/A | N/A | N/A | 664 |
| 58 | 29995 | 1 | MMA | 100 | N/A | N/A | N/A | N/A | 800 |
| 59 | 29995 | 5 | MMA | 100 | N/A | N/A | N/A | N/A | 664 |
| 60 | 42125 | 1 | MMA | 100 | N/A | N/A | N/A | N/A | 391 |
| 61 | 42125 | 1 | MMA | 100 | N/A | N/A | N/A | N/A | 800 |
| 62 | 42125 | 5 | MMA | 100 | N/A | N/A | N/A | N/A | 323 |
| 63 | 50629 | 1 | MMA | 100 | N/A | N/A | N/A | N/A | 595 |
| 64 | 42125 | 1 | DMAEMA | 15 | MMA | 70 | MAA | 15 | 400 |
| 65 | 42125 | 1 | DMAEMA | 25 | MMA | 70 | MAA | 5 | 400 |
| 66 | 58681 | 1 | DMAEMA | 15 | MMA | 55 | MAA | 30 | 200 |
| 67 | 47481 | 1 | MMA | 100 | N/A | N/A | N/A | N/A | 50 |
| 68 | 47481 | 1 | MMA | 100 | N/A | N/A | N/A | N/A | 450 |
| 69 | 9919 | 1 | MMA | 100 | N/A | N/A | N/A | N/A | 100 |
| 70 | 9919 | 1 | MMA | 100 | N/A | N/A | N/A | N/A | 500 |
| 71 | 26262 | 1 | MMA | 100 | N/A | N/A | N/A | N/A | 500 |
| 72 | 34422 | 1 | MMA | 100 | N/A | N/A | N/A | N/A | 500 |
| 73 | 34422 | 1 | MMA | 100 | N/A | N/A | N/A | N/A | 150 |
| 74 | 56390 | 1 | MMA | 100 | N/A | N/A | N/A | N/A | 150 |
| 75 | 9919 | 1 | MMA | 100 | N/A | N/A | N/A | N/A | 550 |
| 76 | 34422 | 1 | MMA | 100 | N/A | N/A | N/A | N/A | 550 |
| 77 | 56390 | 1 | MMA | 100 | N/A | N/A | N/A | N/A | 200 |
| 78 | 34422 | 1 | MMA | 100 | N/A | N/A | N/A | N/A | 600 |
| 79 | 9919 | 1 | MMA | 100 | N/A | N/A | N/A | N/A | 250 |
| 80 | 26262 | 1 | MMA | 100 | N/A | N/A | N/A | N/A | 250 |
| 81 | 34422 | 1 | MMA | 100 | N/A | N/A | N/A | N/A | 250 |
| 82 | 56390 | 1 | MMA | 100 | N/A | N/A | N/A | N/A | 250 |
| 83 | 26262 | 1 | MMA | 100 | N/A | N/A | N/A | N/A | 650 |
| 84 | 47481 | 1 | MMA | 100 | N/A | N/A | N/A | N/A | 650 |
| 85 | 47481 | 1 | MMA | 100 | N/A | N/A | N/A | N/A | 700 |
| 86 | 26262 | 1 | MMA | 100 | N/A | N/A | N/A | N/A | 350 |
| 87 | 26262 | 1 | MMA | 100 | N/A | N/A | N/A | N/A | 750 |
| 88 | 56390 | 1 | MMA | 100 | N/A | N/A | N/A | N/A | 750 |
| 89 | 32669 | 1 | DMAEMA | 20 | MMA | 50 | EAA | 30 | 800 |
| 90 | 37962 | 1 | DMAEMA | 10 | MMA | 70 | EAA | 20 | 200 |
| 91 | 37962 | 1 | DMAEMA | 20 | MMA | 50 | EAA | 30 | 200 |
| 92 | 37962 | 1 | DMAEMA | 20 | MMA | 60 | EAA | 20 | 200 |
| 93 | 50311 | 1 | DMAEMA | 10 | MMA | 80 | EAA | 10 | 50 |
| 94 | 37962 | 1 | MMA | 70 | MAA | 30 | N/A | N/A | 400 |
| 95 | 37962 | 1 | MMA | 70 | PEGMA | 30 | N/A | N/A | 200 |
| 96 | 43305 | 1 | MMA | 70 | PEGMA | 30 | N/A | N/A | 200 |
| 97 | 50311 | 1 | MMA | 70 | PEGMA | 30 | N/A | N/A | 200 |
| 98 | 37962 | 1 | MMA | 80 | PEGMA | 20 | N/A | N/A | 300 |
| 99 | 43305 | 1 | MMA | 70 | PEGMA | 30 | N/A | N/A | 300 |

^{a}Degree of Polymerization
Block 1 is a DMAEMA homopolymer for PNPs 1-66 and 89-99. Block 1 is an AEMA homopolymer for PNPs 67-88.

### Example 3: Nanoparticle Characterization

### Polymer Nanoparticle Size and Polydispersity

Lyophilized polymers were rehydrated with MilliQ water followed by shaking at 300 rpm on an orbital shaker for 2 hours, sonicated in a bath sonicator for 25 minutes, and vortexed for 15 minutes. Then, the samples were centrifuged at 2000 rpm for 5 minutes to remove large aggregates and dust from the sample solutions. Samples of 20 μL were aliquoted into a 96- or 384-well plate that approved for use with the Wyatt Technology, Inc. DynaPro Plate Reader III (DynaPro), and 180 μL of diluent were added to each well. The plate was subsequently sealed with a clear plate cover, and the plate inspected for dust or scratches prior to placement in the DynaPro. Once the plate was placed in the instrument, the software was prepared to record 7 DLS acquisitions of 5 seconds each for each well being measured. The temperature was held constant at 25° C. Once recorded, the data was filtered using the data filtering options within the software, and exported for further review and analysis. Sample measurements were conducted in triplicate, the average and standard deviation values were used to assess the polymer nanoparticle size.

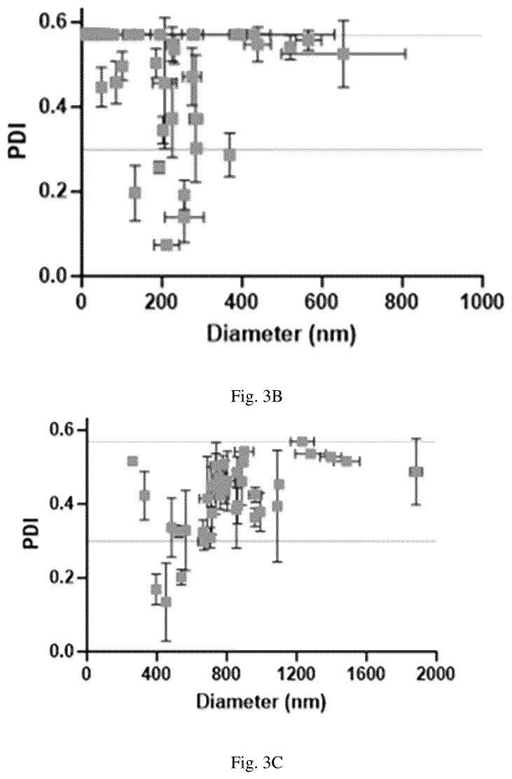

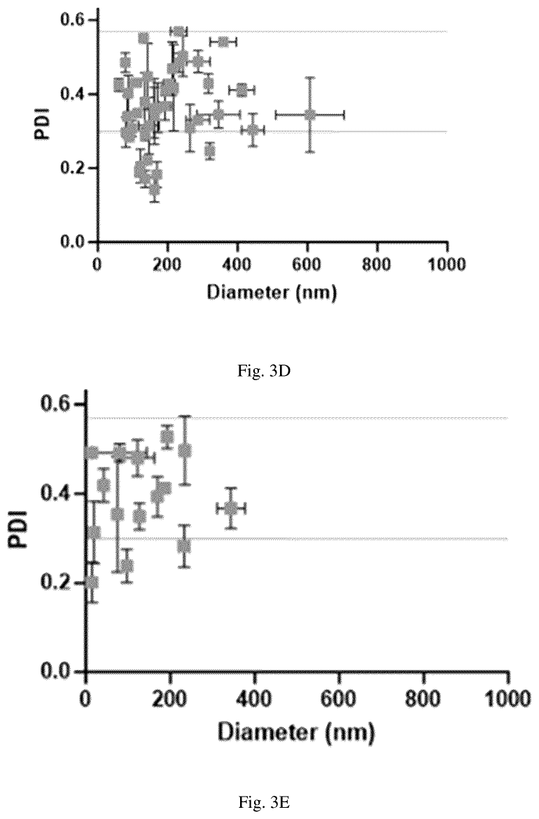

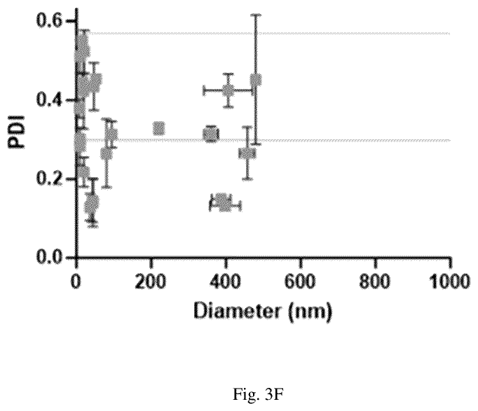

PDI and diameter results are shown in FIGS. 3A-3F. The first (FIG. 3A) library included a first block having a DMAEMA homopolymer and a second block having an MMA homopolymer. The second library (FIG. 3B) library included a first block having a DMAEMA homopolymer and a second block having a DMAEMA, MMA, and MAA copolymer. The third library (FIG. 3C) included a first block having an AEMA homopolymer and a second block having an MMA homopolymer. The fourth library (FIG. 3D) included a first block having a DMAEMA homopolymer and a second block having a DMAEMA, MMA, and EAA copolymer. The fifth library (FIG. 3E) included a first block having a DMAEMA homopolymer and a second block having an MMA and MAA copolymer. The sixth library (FIG. 3F) included a first block having a DMAEMA homopolymer and a second block having an MMA and PEGMA copolymer.

PNPs made above (i.e., PNPs 1-99) have diameter and PDI values as shown in Table 7.

TABLE 7
Size and PDI of PNPs 1-99

Size

| PNP ID | Diameter | |
|---|---|---|
| ID | (nm) | PDI |
| 1 | 236 | 0.159 |
| 2 | 255 | 0.125 |
| 3 | 317 | 0.232 |
| 4 | 238 | 0.139 |
| 5 | 254 | 0.297 |
| 6 | 191 | 0.325 |
| 7 | 75 | 0.201 |
| 8 | 214 | 0.158 |
| 9 | 196 | 0.143 |
| 10 | 258 | 0.297 |
| 11 | 252 | 0.174 |
| 12 | 267 | 0.228 |
| 13 | 184 | 0.49 |
| 14 | 256 | 0.571 |
| 15 | 658 | 0.571 |
| 16 | 393 | 0.311 |
| 17 | 371 | 0.571 |
| 18 | 230 | 0.153 |
| 19 | 278 | 0.319 |
| 20 | 167 | 0.247 |
| 21 | 162 | 0.362 |
| 22 | 85 | 0.571 |
| 23 | 227 | 0.128 |
| 24 | 203 | 0.164 |
| 25 | 299 | 0.283 |
| 26 | N/A | N/A |
| 27 | 194 | 0.381 |
| 28 | 145 | 0.204 |
| 29 | 361 | 0.455 |
| 30 | 196 | 0.211 |
| 31 | 421 | 0.419 |
| 32 | 189 | 0.148 |
| 33 | 192 | 0.571 |
| 34 | 75 | 0.28 |
| 35 | 65 | 0.476 |
| 36 | 195 | 0.12 |
| 37 | 184 | 0.141 |
| 38 | 211 | 0.131 |
| 39 | 135 | 0.225 |
| 40 | 311 | 0.377 |
| 41 | 233 | 0.314 |
| 42 | 384 | 0.571 |
| 43 | 215 | 0.134 |
| 44 | 194 | 0.262 |
| 45 | 345 | 0.397 |
| 46 | 214 | 0.335 |
| 47 | 294 | 0.448 |
| 48 | 207 | 0.207 |
| 49 | 203 | 0.224 |
| 50 | 241 | 0.355 |
| 51 | 328 | 0.244 |
| 52 | 336 | 0.336 |
| 53 | 303 | 0.422 |
| 54 | 330 | 0.390 |
| 55 | 304 | 0.486 |
| 56 | 352 | 0.571 |
| 57 | 306 | 0.514 |
| 58 | 381 | 0.543 |
| 59 | 385 | 0.571 |
| 60 | 282 | 0.426 |
| 61 | 330 | 0.571 |
| 62 | 282 | 0.214 |
| 63 | 249 | 0.229 |
| 64 | 276 | 0.571 |
| 65 | 385 | 0.571 |
| 66 | 23 | 0.571 |
| 67 | 37 | 0.571 |
| 68 | 740 | 0.502 |
| 69 | 376 | 0.571 |
| 70 | 355 | 0.571 |
| 71 | 689 | 0.415 |
| 72 | 962 | 0.364 |
| 73 | 707 | 0.396 |
| 74 | 763 | 0.423 |
| 75 | 298 | 0.571 |
| 76 | 1089 | 0.298 |
| 77 | 871 | 0.571 |
| 78 | 992 | 0.378 |
| 79 | 295 | 0.571 |
| 80 | 670 | 0.298 |
| 81 | 1101 | 0.453 |
| 82 | 859 | 0.514 |
| 83 | 589 | 0.571 |
| 84 | 1394 | 0.556 |
| 85 | 1913 | 0.571 |
| 86 | 862 | 0.571 |
| 87 | 932 | 0.571 |
| 88 | 1884 | 0.515 |
| 89 | 229 | 0.571 |
| 90 | 466 | 0.571 |
| 91 | 346 | 0.571 |
| 92 | 251 | 0.571 |
| 93 | 87 | 0.402 |
| 94 | 192 | 0.367 |
| 95 | 18 | 0.571 |
| 96 | 80 | 0.571 |
| 97 | 19 | 0.571 |
| 98 | 148 | 0.571 |
| 99 | 398 | 0.571 |

### Example 4: Immunogenicity

The Griess Assay evaluation was completed according to the Promega G2930 Griess Reagent System protocol. Briefly, a standard curve of nitrate solution was prepared and added to the designated wells within a multiwell plate. Each well in the standard curve recieved a total of 50 μL of solution. Then, 50 μL of supernatant media from cell culture was added to the wells designated for experimental samples. Next, 50 μL room temperature Sulfanilamide Solution was added to all standard and experimental wells after incubating the plate in the dark for 5-10 minutes at room temperature. 50 μL of room temperature NED solution was added to all standard and experimental wells. Subsequently, the plate was incubated again in the dark for 5-10 minutes at room temperature. A purple/magenta color formed immediately. The absorbance of each solution was measured at 535 nm within 30 minutes, and the experimental concentrations were determined based on the standard curve results. Immunogenicity results of polymer nanoparticles (PNPs) loaded with model pDNA cargo at a 15:1 mass ratio obtained via Griess Assay evaluation are shown in Table 8.

TABLE 8
Immunogencity of PNPs 52-59

| PNP | |
|---|---|
| ID | Griess Assay Absorbance |
| ID | 15:1 |
| 52 | 0.059 |
| 53 | 0.059 |
| 54 | 0.061 |
| 55 | 0.063 |
| 56 | 0.065 |
| 57 | 0.057 |
| 58 | 0.058 |
| 59 | 0.060 |
| 60 | 0.057 |
| 61 | 0.058 |
| 62 | 0.055 |
| 63 | 0.056 |
| 64 | 0.056 |
| 65 | 0.060 |
| 66 | 0.057 |
| 67 | 0.131 |
| 68 | 0.128 |
| 69 | 0.136 |
| 70 | 0.058 |
| 71 | 0.059 |
| 72 | 0.059 |
| 73 | 0.062 |
| 74 | 0.055 |
| 75 | 0.055 |
| 76 | 0.060 |
| 77 | 0.055 |
| 78 | 0.063 |
| 79 | 0.057 |
| 80 | 0.060 |
| 81 | 0.055 |
| 82 | 0.053 |
| 83 | 0.060 |
| 84 | 0.058 |
| 85 | 0.059 |
| 86 | 0.062 |
| 87 | 0.054 |
| 88 | 0.059 |
| 89 | 0.055 |
| 90 | 0.075 |
| 91 | 0.074 |
| 92 | 0.058 |
| 93 | 0.053 |
| 94 | 0.052 |
| 95 | 0.130 |
| 96 | 0.130 |
| 97 | 0.134 |
| 98 | 0.126 |
| 99 | 0.124 |

### Example 5: Loading Efficiency

PNPs were screened using a Ribogreen fluorescence loading assay and a model pDNA payload (˜12 kb) to evaluate how well the PNPs would load pDNA cargo. The Ribogreen assay consisted of diluting PNPs to 0.2 mg/mL in 50-100 μL 1× DPBS, diluting the pDNA 1:10, using a multichannel pipette to combine the PNP sample with 1× DPBS and pDNA in a multiwell plate, centrifuging the plate at 300×g for 1 minute and then incubating at room temperature for 30 minutes. During this incubation process, the Ribogreen working solution was prepared by diluting 20× TE buffer to 1× TE buffer in a light-protected 50 mL conical tube. Then, 100 μL of Ribogreen working solution was added to each well, and the plate was incubated at room temperature for 2-5 minutes. Fluorescence measurements (ex. 480 nm, em. 520 nm) were taken, and the data analyzed to determine the loading efficiency based on the standard curve values. The results are shown in Table 9.

TABLE 9
pDNA loading efficiency of PNPs
1-99 at different PNP:DNA ratios.

| | pDNA Loading Efficiency (%) | | |
|---|---|---|---|
| PNP | 10:1 | 15:1 | 30:1 |
| ID | (PNP:DNA) | (PNP:DNA) | (PNP:DNA) |
| 1 | 88.3 | 88.0 | 92.7 |
| 2 | 101.3 | 95.7 | 87.0 |
| 3 | 67.0 | 100.0 | 106.0 |
| 4 | 88.0 | 94.7 | 98.7 |
| 5 | 92.0 | 93.0 | 95.3 |
| 6 | 88.7 | 88.3 | 91.7 |
| 7 | 90.3 | 96.3 | 96.0 |
| 8 | 94.7 | 93.0 | 100.3 |
| 9 | 93.7 | 95.3 | 100.7 |
| 10 | 92.3 | 94.0 | 97.7 |
| 11 | 94.7 | 98.0 | 105.0 |
| 12 | 93.3 | 92.3 | 92.3 |
| 13 | 92.7 | 93.3 | 95.7 |
| 14 | N/A | N/A | N/A |
| 15 | N/A | N/A | N/A |
| 16 | N/A | N/A | N/A |
| 17 | N/A | N/A | N/A |
| 18 | 93.3 | 92.3 | 95.3 |
| 19 | N/A | N/A | N/A |
| 20 | N/A | N/A | N/A |
| 21 | N/A | N/A | N/A |
| 22 | N/A | N/A | N/A |
| 23 | N/A | N/A | N/A |
| 24 | N/A | N/A | N/A |
| 25 | N/A | N/A | N/A |
| 26 | N/A | N/A | N/A |
| 27 | N/A | N/A | N/A |
| 28 | N/A | N/A | N/A |
| 29 | N/A | N/A | N/A |
| 30 | N/A | N/A | N/A |
| 31 | N/A | N/A | N/A |
| 32 | 89.3 | 91.7 | 88.3 |
| 33 | N/A | N/A | N/A |
| 34 | N/A | N/A | N/A |
| 35 | N/A | N/A | N/A |
| 36 | N/A | N/A | N/A |
| 37 | N/A | N/A | N/A |
| 38 | N/A | N/A | N/A |
| 39 | N/A | N/A | N/A |
| 40 | N/A | N/A | N/A |
| 41 | N/A | N/A | N/A |
| 42 | N/A | N/A | N/A |
| 43 | 91.7 | 93.3 | 93.3 |
| 44 | N/A | N/A | N/A |
| 45 | N/A | N/A | N/A |
| 46 | N/A | N/A | N/A |
| 47 | N/A | N/A | N/A |
| 48 | 93.3 | 92.0 | 93.3 |
| 49 | 94.3 | 93.3 | 95.3 |
| 50 | 97.7 | 93.7 | 98.3 |
| 51 | 93.7 | 96.3 | 95.7 |
| 52 | 72.2 | N/A | N/A |
| 53 | 75.1 | N/A | N/A |
| 54 | 79.1 | N/A | N/A |
| 55 | 77.2 | N/A | N/A |
| 56 | 79.1 | N/A | N/A |
| 57 | 77.2 | N/A | N/A |
| 58 | 82.0 | N/A | N/A |
| 59 | 79.9 | N/A | N/A |
| 60 | 83.4 | N/A | N/A |
| 61 | 83.9 | N/A | N/A |
| 62 | 85.1 | N/A | N/A |
| 63 | 83.6 | N/A | N/A |
| 64 | 95.9 | N/A | N/A |
| 65 | 96.8 | N/A | N/A |
| 66 | 96.3 | N/A | N/A |
| 67 | 99.4 | N/A | N/A |
| 68 | 99.3 | N/A | N/A |
| 69 | 99.3 | N/A | N/A |
| 70 | 97.3 | N/A | N/A |
| 71 | 95.2 | N/A | N/A |
| 72 | 95.4 | N/A | N/A |
| 73 | 95.8 | N/A | N/A |
| 74 | 97.2 | N/A | N/A |
| 75 | 92.2 | N/A | N/A |
| 76 | 95.3 | N/A | N/A |
| 77 | 91.7 | N/A | N/A |
| 78 | 94.0 | N/A | N/A |
| 79 | 94.3 | N/A | N/A |
| 80 | 96.1 | N/A | N/A |
| 81 | 94.4 | N/A | N/A |
| 82 | 93.8 | N/A | N/A |
| 83 | 94.6 | N/A | N/A |
| 84 | 94.6 | N/A | N/A |
| 85 | 93.4 | N/A | N/A |
| 86 | 95.1 | N/A | N/A |
| 87 | 94.3 | N/A | N/A |
| 88 | 95.4 | N/A | N/A |
| 89 | 97.3 | N/A | N/A |
| 90 | 96.9 | N/A | N/A |
| 91 | 96.8 | N/A | N/A |
| 92 | 97.1 | N/A | N/A |
| 93 | 97.2 | N/A | N/A |
| 94 | 96.0 | N/A | N/A |
| 95 | 95.6 | N/A | N/A |
| 96 | 95.2 | N/A | N/A |
| 97 | 94.8 | N/A | N/A |
| 98 | 92.2 | N/A | N/A |
| 99 | 95.0 | N/A | N/A |

### Example 6: Transfection Efficiency

Mouse Schwann cells (ABM cell line 1970C₃) were cultured in 96-well plates to allow for 25,000 cells per well. PNP test articles was formulated with the model pDNA payload that expresses tdTomato. 1 day prior to experimentation, multiple PNP:pDNA ratios (e.g., 10:1, etc.) were prepared. Then, the PNP test articles were combined with 1× DPBS in a 96-well plate according to the experimental design. The plates were subsequently centrifuged at 300×g for 1 min and incubated at room temperature for 20 minutes. Controls, such as Lipofectamine 3000 (prepared as per manufacturer's instructions), were also tested with the experimental samples. Following incubation, 10 μL of each sample solution was added directly to the corresponding well of the multiwell plate containing the cultured Schwann cells, and the cell culture plate was returned to the incubator for 48 hours. Once the incubation period ended, the cell culture samples were prepared for flow cytometry using a live/dead stain (e.g., GFP or Zombie Violet), assessment gates were set, and at least 30,000 events were recorded by a CyotFlex flow cytometer. The results were analyzed to determine the transfection efficiency based on the number of events (i.e., cells expressing model tdTomato payload) appeared in the tdTomato positive gate. The results are shown in Table 10.

### Example 7: Cell Viability

Mouse Schwann cells (ABM cell line 1970C3) were cultured in 96-well plates to ensure 25,000 cells per well. PNP test articles were formulated with the model pDNA payload expressing tdTomato with multiple PNP:pDNA ratios (e.g., 10:1, etc.) 1 day prior to the experiment. The following day, the PNP test articles were combined with 1× DPBS in a new 96-well plate according to the experimental design. The plates were centrifuged at 300×g for 1 min and incubated at room temperature for 20 minutes. Controls, such as Lipofectamine 3000 (prepared per manufacturer's instructions), were also tested at the same time as the experimental samples. Following incubation, 10 μL of each sample solution was added directly to the corresponding well of the multiwell plate containing the cultured Schwann cells, and the cell culture plate returned to the incubator for 48 hours. Once the incubation period ended, the cell culture samples were prepared for flow cytometry using a live/dead stain (e.g., Zombie Violet), the assessment gates were set as in Example 6, and at least 30,000 events were recorded by a CyotFlex flow cytometer. The results were analyzed to determine the cell viability based on how many events (i.e., cells expressing the live/dead stain, such as Zombie Violet) appeared in the Zombie Violet positive gate compared to the Zombie Violet negative gate. The results are shown in Table 10. The polymer nanoparticles were well-tolerated by the Schwann cells. Multiple polymer nanoparticles significantly outperformed the Lipofectamine positive control, the gold standard in the field for comparing transfection efficiency.

TABLE 10
Transfection efficiency and cell viability of PNPs 1-99

| PNP | Schwann Cell Transfection Efficiency (%) | | Cell Viability % (Schwann Cells) | |
|---|---|---|---|---|
| ID | 10:1 (PNP:DNA) | 15:1 (PNP:DNA) | 10:1 (PNP:DNA) | 15:1 (PNP:DNA) |
| 1 | 1.3 | 6.5 | 98.0 | 96.4 |
| 2 | 10.3 | 0.2 | 93.4 | 99.5 |
| 3 | 1.0 | 4.3 | 99.3 | 97.5 |
| 4 | 9.7 | 9.9 | 76.5 | 90.7 |
| 5 | 9.3 | 15.3 | 75.5 | 44.7 |
| 6 | 1.1 | 5.1 | 98.9 | 97.9 |
| 7 | 8.3 | 9.7 | 96.1 | 93.8 |
| 8 | 5.9 | 11.2 | 80.8 | 87.4 |
| 9 | 9.2 | 10.4 | 25.3 | 59.7 |
| 10 | 16.5 | 16.5 | 83.2 | 82.8 |
| 11 | 4.7 | 13.3 | 94.5 | 95.1 |
| 12 | 1.4 | 7.2 | 98.8 | 94.3 |
| 13 | 5.0 | 0.1 | 97.5 | 99.6 |
| 14 | N/A | N/A | N/A | N/A |
| 15 | N/A | N/A | N/A | N/A |
| 16 | N/A | N/A | N/A | N/A |
| 17 | N/A | N/A | N/A | N/A |
| 18 | 8.7 | 14.7 | 72.5 | 50.1 |
| 19 | N/A | N/A | N/A | N/A |
| 20 | N/A | N/A | N/A | N/A |
| 21 | N/A | N/A | N/A | N/A |
| 22 | N/A | N/A | N/A | N/A |
| 23 | N/A | N/A | N/A | N/A |
| 24 | N/A | N/A | N/A | N/A |
| 25 | N/A | N/A | N/A | N/A |
| 26 | N/A | N/A | N/A | N/A |
| 27 | N/A | N/A | N/A | N/A |
| 28 | N/A | N/A | N/A | N/A |
| 29 | N/A | N/A | N/A | N/A |
| 30 | N/A | N/A | N/A | N/A |
| 31 | N/A | N/A | N/A | N/A |
| 32 | 13.0 | 13.4 | 72.6 | 48.2 |
| 33 | N/A | N/A | N/A | N/A |
| 34 | N/A | N/A | N/A | N/A |
| 35 | N/A | N/A | N/A | N/A |
| 36 | N/A | N/A | N/A | N/A |
| 37 | N/A | N/A | N/A | N/A |
| 38 | N/A | N/A | N/A | N/A |
| 39 | N/A | N/A | N/A | N/A |
| 40 | N/A | N/A | N/A | N/A |
| 41 | N/A | N/A | N/A | N/A |
| 42 | N/A | N/A | N/A | N/A |
| 43 | 8.6 | 10.3 | 89.8 | 84.6 |
| 44 | N/A | N/A | N/A | N/A |
| 45 | N/A | N/A | N/A | N/A |
| 46 | N/A | N/A | N/A | N/A |
| 47 | N/A | N/A | N/A | N/A |
| 48 | 7.2 | 0.1 | 94.0 | 99.5 |
| 49 | 11.4 | 0.1 | 95.2 | 99.4 |
| 50 | 0.1 | 4.9 | 99.5 | 97.8 |
| 51 | 2.4 | 4.3 | 97.6 | 95.1 |
| 52 | N/A | 0.6 | N/A | 44.1 |
| 53 | N/A | 0.2 | N/A | 56.5 |
| 54 | N/A | 0.7 | N/A | 30.0 |
| 55 | N/A | 0.8 | N/A | 8.9 |
| 56 | N/A | 0.0 | N/A | 36.3 |
| 57 | N/A | 2.6 | N/A | 6.4 |
| 58 | N/A | 0.8 | N/A | 7.8 |
| 59 | N/A | 0.2 | N/A | 24.6 |
| 60 | N/A | 0.4 | N/A | 10.1 |
| 61 | N/A | 0.2 | N/A | 42.0 |
| 62 | N/A | 0.2 | N/A | 29.2 |
| 63 | N/A | 0.9 | N/A | 3.5 |
| 64 | N/A | 1.8 | N/A | 42.7 |
| 65 | N/A | 2.0 | N/A | 38.8 |
| 66 | N/A | 3.8 | N/A | 52.9 |
| 67 | N/A | 0.1 | N/A | 99.7 |
| 68 | N/A | 0.2 | N/A | 99.6 |
| 69 | N/A | 0.1 | N/A | 99.4 |
| 70 | N/A | 0.1 | N/A | 91.2 |
| 71 | N/A | 0.2 | N/A | 71.1 |
| 72 | N/A | 0.2 | N/A | 83.5 |
| 73 | N/A | 0.2 | N/A | 98.5 |
| 74 | N/A | 0.1 | N/A | 85.1 |
| 75 | N/A | 0.1 | N/A | 92.7 |
| 76 | N/A | 0.1 | N/A | 99.5 |
| 77 | N/A | 0.3 | N/A | 86.8 |
| 78 | N/A | 0.0 | N/A | 99.5 |
| 79 | N/A | 0.0 | N/A | 99.8 |
| 80 | N/A | 0.0 | N/A | 87.7 |
| 81 | N/A | 0.1 | N/A | 99.5 |
| 82 | N/A | 0.2 | N/A | 57.2 |
| 83 | N/A | 0.0 | N/A | 99.8 |
| 84 | N/A | 0.7 | N/A | 19.9 |
| 85 | N/A | 0.1 | N/A | 99.1 |
| 86 | N/A | 0.0 | N/A | 25.5 |
| 87 | N/A | 0.1 | N/A | 92.9 |
| 88 | N/A | 0.2 | N/A | 86.6 |
| 89 | N/A | 2.0 | N/A | 36.9 |
| 90 | N/A | 1.4 | N/A | 69.9 |
| 91 | N/A | 0.6 | N/A | 79.6 |
| 92 | N/A | 0.2 | N/A | 97.7 |
| 93 | N/A | 0.2 | N/A | 96.2 |
| 94 | N/A | 0.9 | N/A | 81.0 |
| 95 | N/A | 0.6 | N/A | 88.6 |
| 96 | N/A | 0.1 | N/A | 99.2 |
| 97 | N/A | 0.3 | N/A | 98.2 |
| 98 | N/A | 0.1 | N/A | 99.3 |
| 99 | N/A | 1.2 | N/A | 98.8 |

### Tissue Distribution

In vivo biodistribution testing consisted of test article (TA) preparation, TA dosing, necropsy, specimen collection, tissue processing, and data analysis. The TA consisted of pooled DNA-barcoded PNPs loaded with a non-targeting Cas9 payload prepared at 1.0 mg/kg or 0.01 mg/kg PNP concentrations. Once prepared, the TA was protected from light and stored at −4° C. until used for dosing animals. Dosing volume was adjusted according to animal body weight using PBS prior to administration. Target dose volume was between 5 and 10 μL per mouse (i.e., 0.33 mL/kg or 0.1 mL/30 g mouse). The TA was delivered via intrathecal injection into wild type C57B1/6 mice. Tissues were collected at 24 hours post-injection and the tissues collected included the sciatic nerve, brain (cortex), blood (processed for serum), and liver. Importantly, no adverse effects were observed and all of the mice dosed survived for the duration of the study. At the time of harvest, tissue specimens were flash frozen using liquid nitrogen. After flash freezing, samples were maintained on dry ice until transferred without thawing to a freezer and stored between −85° C. to −60° C. The tissue specimens were then homogenized and DNA was extracted to allow for identification of which DNA barcodes were present in each tissue, thereby indicating which PNPs were present in each tissue. Nucleic acid concentration was quantified using a Nanodrop spectrophotometer. Sequencing to identify barcodes was performed using a MiSeq system. Quantitative polymerase chain reaction (qPCR) was used to determine barcode counts in each tissue, and the barcode identities were matched with the barcode counts to determine the quantitative barcode count results for each PNP tested.

Once the tissues were collected and processed, the statistical analysis tool DESeq2 was used to analyze the biodistribution data by comparing the dosed group to the saline group for each dose and tissue independently. Then, a heuristic approach was used to down-select PNPs of interest. The selection applies a set of filters to identify PNPs that are detected with statistical significance in tissues of interest. Table 11 shows the filtering approach used. The value x is chosen based on the tissue of interest due to differences in sample preparation (tissue mass, PCR cycles, NGS read depth, etc.). The metric p-adjusted is the p-value of the DESeq2 statistical test adjusted for the multiple tests that are required for each PNP in the test article pool. This adjustment keeps the false alarm rate consistent with the desired significance level. Base mean is the mean number of reads after adjusting the raw data for NGS batch effects, bio replicate variation, and outliers. Log2 fold change is the log 2 transformed fold change of the dosed samples relative to the saline samples. Finally, normalized base mean is the base mean adjusted for the unequal distribution of the PNPs in the test article pool.

TABLE 11
Filtering approach used to analyze
in vivo biodistribution study data
Filter Applied

p-adjusted <0.05
Base mean >x where x is 10
Log2 fold change >0
Normalized base mean Sort descending

The tissue distribution results for PNPs 1-99 after applying the filters in Table 11 are shown in Table 12.

TABLE 12
Biodistribution results for PNPs 1-99 in mice

| PNP | |
|---|---|
| ID | |
| ID | Tissue Distribution |
| 1 | Brain, Sciatic Nerve |
| 2 | Brain, Kidney, Liver, Lung, Sciatic |
| | Nerve |
| 3 | Brain, Kidney, Liver, Lung |
| 4 | Sciatic Nerve |
| 5 | Sciatic Nerve |
| 6 | Lung, Sciatic Nerve |
| 7 | Sciatic Nerve |
| 8 | Kidney, Liver, Lung |
| 9 | Lung |
| 10 | Lung |
| 11 | Lung |
| 12 | Lung |
| 13 | Lung |
| 14 | N/A |
| 15 | N/A |
| 16 | N/A |
| 17 | N/A |
| 18 | N/A |
| 19 | N/A |
| 20 | N/A |
| 21 | N/A |
| 22 | N/A |
| 23 | N/A |
| 24 | N/A |
| 25 | N/A |
| 26 | N/A |
| 27 | N/A |
| 28 | N/A |
| 29 | N/A |
| 30 | N/A |
| 31 | N/A |
| 32 | N/A |
| 33 | N/A |
| 34 | N/A |
| 35 | N/A |
| 36 | N/A |
| 37 | N/A |
| 38 | N/A |
| 39 | N/A |
| 40 | N/A |
| 41 | N/A |
| 42 | N/A |
| 43 | N/A |
| 44 | N/A |
| 45 | N/A |
| 46 | N/A |
| 47 | N/A |
| 48 | N/A |
| 49 | N/A |
| 50 | N/A |
| 51 | N/A |
| 52 | Brain |
| 53 | Brain, Sciatic Nerve |
| 54 | Sciatic Nerve |
| 55 | Sciatic Nerve |
| 56 | Sciatic Nerve |
| 57 | Brain |
| 58 | Liver |
| 59 | Brain, Sciatic Nerve |
| 60 | Brain |
| 61 | Brain |
| 62 | Brain |
| 63 | Brain |
| 64 | Brain |
| 65 | Brain |
| 66 | Brain |
| 67 | Sciatic Nerve |
| 68 | Sciatic Nerve |
| 69 | Sciatic Nerve |
| 70 | Sciatic Nerve |
| 71 | Sciatic Nerve |
| 72 | Sciatic Nerve |
| 73 | Brain, Sciatic Nerve |
| 74 | Sciatic Nerve |
| 75 | Sciatic Nerve |
| 76 | Sciatic Nerve |
| 77 | Sciatic Nerve |
| 78 | Sciatic Nerve |
| 79 | Sciatic Nerve |
| 80 | Sciatic Nerve |
| 81 | Sciatic Nerve |
| 82 | Sciatic Nerve |
| 83 | Sciatic Nerve |
| 84 | Sciatic Nerve |
| 85 | Sciatic Nerve |
| 86 | Sciatic Nerve |
| 87 | Brain, Sciatic Nerve |
| 88 | Sciatic Nerve |
| 89 | Sciatic Nerve |
| 90 | Brain |
| 91 | Brain |
| 92 | Brain |
| 93 | Sciatic Nerve |
| 94 | Brain |
| 95 | Sciatic Nerve |
| 96 | Sciatic Nerve |
| 97 | Sciatic Nerve |
| 98 | Sciatic Nerve |
| 99 | Brain |

N/A = not found in tissue

1. A block copolymer comprising:
a first block homopolymer of monomers, wherein each monomer unit is represented by formula I: 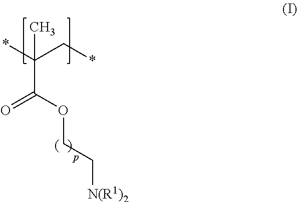
or a salt thereof, wherein:
p is 0 or an integer selected from 1-3,
each R¹ is individually selected from H or C₁-C₆ alkyl; and
wherein each * individually represents a point of covalent attachment to the rest of the block copolymer; and
a second block of either: (i) a homopolymer of monomer units, wherein each monomer unit is represented by formula II: 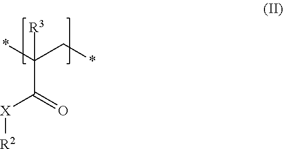
or a salt thereof, wherein:
X is —O— or —NH—;
R² is H or C₁-C₆ alkyl; and
R³ is C₁-C₃ alkyl; or
(ii) a copolymer comprising a copolymer of two or more monomer units each individually represented by formula II: 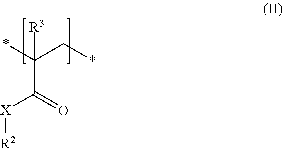
or a salt thereof, wherein:
X is —O— or —NR²—;
each R² is H, C₁-C₆ alkyl, or polyethylene glycol, wherein each hydrogen in C₁-C₆ alkyl is optionally substituted by amino; and
R³ is C₁-C₃ alkyl;
wherein each * individually represents a point of covalent attachment to the rest of the block copolymer; and
wherein the first block has a molecular weight (Mw) of at least 9,000 Da.

2. The block copolymer of claim 1, wherein p is 1.

3. (canceled)

4. The block copolymer of claim 2, wherein each R¹ is methyl.

5. (canceled)

6. (canceled)

7. The block copolymer of claim 1, wherein the second block comprises a homopolymer of a monomer unit represented by formula II.

8. The block copolymer of claim 7, wherein X is —O—.

9. (canceled)

10. (canceled)

11. The block copolymer of claim 8, wherein R² is methyl.

12. The block copolymer of claim 1, wherein the second block comprises a copolymer comprising a first monomer unit represented by formula II and a second monomer unit represented by formula II.

13. (canceled)

14. (canceled)

15. The block copolymer of claim 12, wherein R² is methyl in the first monomer unit.

16. The block copolymer of claim 12, wherein R² is butyl in the first monomer unit.

17. (canceled)

18. The block copolymer of claim 12, wherein R² is H in the second monomer unit.

19. The block copolymer of claim 12, wherein R² is polyethylene glycol in the second monomer unit.

20-26. (canceled)

27. The block copolymer of claim 1, wherein each R³ is methyl.

28. (canceled)

29. A block copolymer comprising a first block and a second block, wherein:
the first block comprise a homopolymer of poly dimethylaminoethyl methacrylate (DMAEMA) or poly aminoethyl methacrylate (AEMA), and
the second block comprises either:
(a) a homopolymer of polymethylmethacrylate (MMA) or polybutylmethacrylate (BMA) or
(b) a copolymer of at least two of DMAEMA, MMA, BMA, polyethyleneglycol methacrylate (PEGMA), methylacrylic acid (MAA), and ethylacrylic acid (EAA),
wherein the first block has a molecular weight (Mw) of at least 9,000 Da.

30. (canceled)

31. (canceled)

32. The block copolymer of claim 1, wherein the first block has a molecular weight (Mw) of about 25,000 Da to about 60,000 Da.

33. The block copolymer of claim 29, wherein the first block has a molecular weight (Mw) of about 25,000 Da to about 50,000 Da.

34. The block copolymer of claim 29, wherein the first block has a molecular weight (Mw) of about 9,000 Da to about 20,000 Da.

35. (canceled)

36. (canceled)

37. The block copolymer of claim 1, wherein the second block has a degree of polymerization of at least 250.

38. (canceled)

39. The block copolymer of claim 1, wherein the second block has a degree of polymerization of about 350 to about 700.

40. The block copolymer of claim 1, wherein the first block comprises a cap of formula: 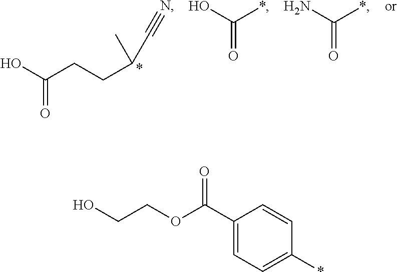
or a salt thereof, wherein * represents a point of covalent attachment to the first block.

41. (canceled)

42. The copolymer of claim 1, wherein the second block comprises a cap of formula: 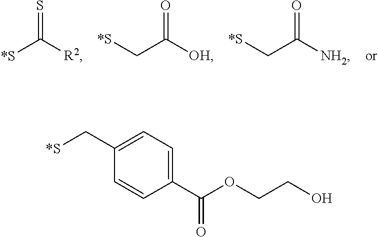
or a salt thereof, wherein * represents a point of covalent attachment to the second block, and R² is —SC₂-C₁₂ alkyl or C₆H₅.

43. (canceled)

44. A polymer nanoparticle comprising:
a block copolymer according to claim 1,
wherein the nanoparticle has a diameter of about 20 nm to about 2000 nm.

45-48. (canceled)

49. The polymer nanoparticle of claim 44, wherein the nanoparticle has a diameter of about 200 nm to about 500 nm.

50. The polymer nanoparticle of claim 44, wherein the polymer nanoparticle has a polydispersity index (PDI) of lower than about 0.3.

51. (canceled)

52. A composition comprising:
a polymer nanoparticle according to claim 44, and
a nucleic acid complexed to the polymer nanoparticle.

53. The composition of claim 52, wherein the polymer nanoparticle is complexed to the nucleic acid via electrostatic interaction.

54. The composition of claim 52, wherein the encapsulation efficiency is at least about 75%.

55. The composition of claim 52, wherein the transfection efficiency of the composition into cells is at least about 1%.

56. (canceled)

57. (canceled)

58. A method of treating a disease in a patient in need thereof, the method comprising:
administering a therapeutically effective amount of a composition according to claim 52.

59. (canceled)

60. The method of claim 58, wherein the disease affects the sciatic nerve.

61. The method of claim 58, wherein the disease affects the brain.

62-64. (canceled)
